(19) United States
(12) Patent Application Publication
(10) Pub. No.: US 2024/0366609 A1
(43) Pub. Date: Nov. 7, 2024

(54) COMBINATION THERAPY WITH BELVARAFENIB AND COBIMETINIB OR WITH BELVARAFENIB, COBIMETINIB, AND ATEZOLIZUMAB

(71) Applicants: Genentech, Inc., South San Francisco, CA (US); Hoffmann-La Roche Inc., Little Falls, NJ (US); Hanmi Pharm. Co., Ltd., Seoul (KR)

(72) Inventors: Maria Suhady ANDERSON, South San Francisco, CA (US); Michael John DOLTON, Sydney (AU); Shiva MALEK, South San Francisco, CA (US); Ehud SEGAL, South San Francisco, CA (US); Vikram MALHI, South San Francisco, CA (US); Jennifer ENG-WONG, South San Francisco, CA (US); Yibing YAN, South San Francisco, CA (US); Ivana Yen Yen YEN, South San Francisco, CA (US); Seungjae BAEK, Seoul (KR)

(21) Appl. No.: 18/554,041

(22) PCT Filed: Apr. 5, 2022
(86) PCT No.: PCT/US22/23496

§ 371 Date: Oct. 5, 2023

Related U.S. Application Data
(60) Provisional application No. 63/171,461, filed on Apr. 6, 2021.

(51) Int. Cl. A61K 31/519; A61K 9/00; A61K 31/4523; A61K 39/395; A61P 35/00

(52) U.S. Cl. CPC A61K 31/519; A61K 9/0053; A61K 31/4523; A61K 39/3955; A61P 35/00

(57) ABSTRACT

Combination therapies comprising belvarafenib and cobimetinib and comprising belvarafenib, cobimetinib, and atezolizumab are provided for the treatment of melanoma carrying a NRAS mutation.

### CROSS REFERENCE TO RELATED APPLICATION

This application claims priority to U.S. Provisional Application Ser. No. 63/171,461 filed on Apr. 6, 2021. The entire text of that provisional application is incorporated by reference into this application.

### FIELD OF THE DISCLOSURE

The field of the invention relates generally to cancer therapy with a combination of belvarafenib and cobimetinib and with a combination of belvarafenib, cobimetinib and atezolizumab for the treatment of NRAS-mutant melanoma.

### BACKGROUND

Melanoma is a potentially deadly form of skin cancer originating from melanocytes. Although the outcome for promptly diagnosed superficial tumors is good, melanoma in the metastatic setting is associated with high rates of mortality and disease related morbidity.

The RAS/RAF/MEK/ERK mitogen-activated protein kinase (MAPK) signaling cascade is a key intracellular signaling network that transduces multiple signals from the extracellular environment to the nucleus of cells to activate cellular growth and differentiation (Johnson G L, Lapadat R. Mitogen-activated protein kinase pathways mediated by ERK, JNK, and p38 protein kinases. Science 2002; 298:1911-2; Roberts P J, Der C J. Targeting the Raf-MEK-ERK mitogen-activated protein kinase cascade for the treatment of cancer. Oncogene 2007; 26:3291-310). This pathway is highly implicated in the pathogenesis of melanoma. Approximately 40% to 50% of melanomas harbor an activating mutation in BRAF (Davies H, Bignell G R, Cox C, et al. Mutations of the BRAF gene in human cancer. Nature 2002, 417:949-54; Curtin J A, Fridyland J, Kageshita T, et al. Distinct sets of genetic alterations in melanoma. N Engl J Med 2005, 353:2135-47; Jakob J A, Bassett Jr R L, Ng C S, et al. NRAS mutation status is an independent prognostic factor in metastatic melanoma. Cancer 2012, 118:4014-23), and 29% of melanomas harbor mutations in NRAS (Moore A R, Rosenberg S C, McCormick F, et al. RAS-targeted therapies: is the undruggable drugged? Nat Rev Drug Discov 2020; 19:533-52).

Melanoma cells are highly immunogenic and thus an appropriate target for immunotherapy (Zhu F., Liang Yu, Chen D, et al. Melanoma antigen gene family in the cancer immunotherapy. Cancer Transl Med 2016; 2:85-9.). The advent of immunotherapy has dramatically shifted the outcomes for patients with melanoma. In the past decade, overall survival (OS) for patients with advanced stage melanoma has improved from 9 months to ≥5 years due to the impact of immunotherapy agents. A number of Phase III trials have compared single-agent anti-PD-1 inhibitors to anti-CTLA4 inhibitors or chemotherapy and have shown improvements in objective response rate (ORR), progression-free survival (PFS), and OS, with an OS of approximately 3 years and PFS rates ranging from 4 to 7 months (Robert C, Schachter J, Long G, et al. Pembrolizumab versus Ipilimumab in Advanced Melanoma. N Engl J Med 2015, 372:2521-32; Schachter J, Ribas A, Long G V. Pembrolizumab versus ipilimumab for advanced melanoma: final overall survival results of a multicentre, randomised, open-label phase 3 study. Lancet 2017, 390, 1853-62). Combination immunotherapy provides an even more robust benefit. In a Phase III trial of 1296 patients, those who were randomized to nivolumab and ipilimumab had an increase in both PFS and OS compared with patients on nivolumab alone. PFS was 11.5 months in the nivolumab plus ipilimumab arm and 6.9 months in the nivolumab monotherapy arm with a hazard ratio (HR) of 0.42 (95% CI: 0.35 to 0.51). At 5 years minimum follow-up, the median OS has not been reached and is >60.0 months (95% CI: 38.2 to “not reached”) for nivolumab plus ipilimumab and is 36.9 months (95% CI: 28.2 to 58.7) for nivolumab alone, with an HR of 0.052 (95% CI: 0.42 to 0.64) (Larkin J, Chiarion-Sileni V, Gonzalez R, et al. Five-year survival with combined nivolumab and ipilimumab in advanced melanoma. N Engl J Med 2019; 381:1535-46). Of note, >50% of patients experienced Grade ≥3 adverse events on nivolumab plus ipilimumab, and treatment was difficult to complete, with 36.4% of patients discontinuing therapy due to adverse events

No Phase III trials have been conducted solely in patients who have disease progression following treatment with anti-PD-1 agents. Patients who received low-dose ipilimumab with pembrolizumab immediately following progression on PD-1 antibody in melanoma showed significant anti-tumor activity in a Phase II trial. In patients who have previously undergone anti-PD-1 treatment, response rates with repeat use of immunotherapy range from approximately 15% for ipilimumab (Robert C, Ribas A, Schachter J, et al. Pembrolizumab versus ipilimumab in advanced melanoma (KEYNOTE-006): post-hoc 5-year results from an open-label, multicentre, randomised, controlled, phase 3 study. Lancet Oncol 2019; 20:1239-51) to 27% (immune-related response criteria) for combination of pembrolizumab and ipilimumab, with PFS of 5 months (Olson D, Luke J, Poklepovic A S, et al. Significant antitumor activity for low-dose ipilimumab (IPI) with pembrolizumab (PEMBRO) immediately following progression on PD1 Ab in melanoma (MEL) in a phase II trial. J Clin Oncol 2020; 38:10004).

Patients with melanoma are further identified for appropriate therapy by mutations in BRAF^{V600}. Patients without a BRAF mutation are collectively referred to as BRAF wild type (WT); these cancers may include melanomas with NRAS mutation, NF1 mutation, and those without any mutation identified or “triple WT.” Approved treatments for patients with BRAF WT melanoma include immunotherapy agents, chemotherapeutic agents, and T-VEC. No targeted therapy has been identified for patients with BRAF WT melanoma. Approved treatments for patients with BRAF-mutant melanoma include targeted therapy (BRAF inhibitor alone or in combination with MEK inhibitors), immunotherapy, and chemotherapy. The optimal treatment sequencing (i.e., targeted therapy followed by immunotherapy or vice versa) for patients with BRAF-mutant tumors is not known.

Within the BRAF WT subset, a number of common mutations have been described, including NRAS mutations, which occur in approximately 29% of all patients with melanoma (Moore et al. 2020). Mutations in NRAS occur either at residue Glycine 12 (G12), Glycine 13 (G13), or Glutamine 61 (Q61). Approximately 85% of NRAS-mutant melanomas harbor mutations in NRAS Q61 (enriched for Q61R, Q61K, Q61L, Q61H) (Moore et al. 2020), with a far smaller fraction of melanomas harboring mutations in NRAS G12 or G13. The most common NRAS non-Q61 mutations are NRAS G12D, G13R, and G13D (Li S, Balmain A, Counter C M. A modelfor RAS mutation patterns in cancers: finding the sweet spot. Nat Rev Cancer 2018; 18:767-7). In a tissue-specific conditional knock-in mouse model of melanoma, expression of NRAS Q61R has been shown to drive melanoma formation, with mechanistic studies demonstrating that the NRAS Q61R mutation exhibits distinct nucleotide-binding capacity, stability, and GTPase resistance likely driving its melanomagenic properties (Burd C E, Liu W, Huynh M V, et al. Mutation-specific RAS oncogenicity explains NRAS codon 61 selection in melanoma. Cancer Discov 2014; 4:1418-29). The importance of MAPK signaling, specifically BRAF and CRAF kinases downstream of RAS in NRAS-mutant melanoma, has also been validated in NRAS^{Q61K}-induced melanoma mouse models, in which conditional ablation of BRAF and CRAF genes resulted in complete blockage of tumor growth (Dorard C, Estrada C, Barbotin C, et al. RAF proteins exert both specific and compensatory functions during tumour progression of NRAS-driven melanoma. Nat Commun 2017; 8:1-13).

Despite the prevalence of NRAS mutations and the severity of the resulting disease, there are few treatment options available.

Treatment of NRAS-mutant melanoma in patients who have disease progression on or after treatment with anti-PD-1 agents represents a significant unmet medical need. New targeted treatment approaches are needed for these patients, who have an identified activating MAPK pathway mutation.

### BRIEF DESCRIPTION

The present disclosure provides a method of treating a subject having NRAS-mutant melanoma.

In some aspects, the method comprises: (i) administering to said subject a therapy consisting essentially of (ii) a therapeutically effective amount of belvarafenib, or a pharmaceutically acceptable salt thereof and (iii) a therapeutically effective amount of cobimetinib, or a pharmaceutically acceptable salt thereof.

In some aspects, the subject is administered: (i) from about 200 mg to about 1300 mg, from about 400 mg to about 1200 mg, from about 600 mg to about 1200 mg, or from about 800 mg to about 1000 mg, of belvarafenib or a pharmaceutically acceptable salt thereof per day and (ii) from about 20 mg to about 100 mg of cobimetinib or a pharmaceutically acceptable salt thereof per day.

In some aspects, the method comprises: (i) administering to the subject a therapy consisting essentially of (ii) a therapeutically effective amount of belvarafenib or a pharmaceutically acceptable salt thereof, (iii) a therapeutically effective amount of cobimetinib or a pharmaceutically acceptable salt thereof, and (iv) a therapeutically effective amount of atezolizumab.

In some aspects, the subject is administered: (i) from about 200 mg to about 1300 mg, from about 400 mg to about 1200 mg, from about 600 mg to about 1200 mg, or from about 800 mg to about 1000 mg, of belvarafenib or a pharmaceutically acceptable salt thereof per day; (ii) from about 20 mg to about 100 mg of cobimetinib or a pharmaceutically acceptable salt thereof per day; and (iii) from about 500 mg to about 2000 mg, from about 500 mg to about 1000 mg, from about 750 mg to about 1000 mg, from about 750 mg to about 2000 mg, from about 1000 mg to about 2000 mg, from about 1500 mg to about 1750 mg of the atezolizumab.

### BRIEF DESCRIPTION OF THE DRAWINGS

FIG. 1 is a diagram of the design study for aspects of the present disclosure directed to the treatment of NRAS-mutant melanoma with a combination of (i) belvarafenib and cobimetinib and (ii) with a combination of belvarafenib, cobimetinib, and atezolizumab. In FIG. 1: A refers to atezolizumab; B refers to belvarafenib; BID refers to twice a day; C refers to cobimetinib dosed 21 of 28 days; DLT refers to dose-limiting toxicity; QD refers to every day; Q4W refers to every 4 weeks; x refers to dose (and schedule) to be determined. Dotted lines indicate de-escalation groups. Further: ^{a} indicates the inclusion of 5 patients who agree to a mandatory serial biopsy at screening; approximately 6 weeks after Cycle 1, Day 1; and on disease progression (if considered safe). Further: ^{b} indicates that up to 25 patients to be enrolled in Arm 2 at same dose of belvarafenib and cobimetinib

FIG. 2A is a depiction of a western blot for in vitro MVEK/ERK/RSK signaling pathway inhibition by belvarafenib (Belv), cobimetinib (Cobi), or their combination in the SK-MEL-30 (NRAS^{Q61K}) mutant melanoma cell line.

FIG. 2B is a depiction of a western blot for in vitro MVEK/ERK/RSK signaling pathway inhibition by belvarafenib (Belv), cobimetinib (Cobi), or their combination in the IPC-298 (NRAS^{Q61L}) mutant melanoma cell line.

FIG. 3 is a depiction of the result of a colony formation assays in the IPC-298 (NRAS^{Q61L}) melanoma cell line treated with belvarafenib, cobimetinib, or their combination.

FIG. 4 is a depiction of the result of a colony formation assays in the Mel-Juso (NRAS^{Q61L}) melanoma cell line treated with belvarafenib, cobimetinib, or their combination.

FIG. 5 is a depiction of the result of a colony formation assays in the SK-MEL-30 (NRAS^{Q61K}) melanoma cell line treated with belvarafenib, cobimetinib, or their combination.

FIG. 6 is a depiction of in vivo tumor volume antitumor activities of belvarafenib (designated as HM95573) or cobimetinib alone and their combination as measured at days 1, 4, 8, 11, and 15 for a regimen where the drugs are administered orally for 14 days in mice xenografted with SK-MEL-30 melanoma cancer cell line.

FIG. 7 is a depiction of mouse body weight change in evaluation of in vivo tumor volume antitumor activities of belvarafenib (designated as HM95573) or cobimetinib alone and their combination as measured at days 1, 4, 8, 11, and 15 for a regimen where the drugs are administered orally for 14 days in mice xenografted with SK-MEL-30 melanoma cancer cell line.

FIG. 8 is a depiction of antitumor activities of belvarafenib (designated as HM95573) or binimetinib (designated as MEK 162) alone and their combination as measured on days 1, 4, 7, 11, 14, 18, and 21 for a regimen where the drugs are administered orally for 21 days in mice xenografted with SK-MEL-30 melanoma cancer cell line.

FIG. 9 is a depiction of body weight change associated with belvarafenib (designated as HM95573) or binimetinib (designated as MEK 162) alone and their combination as measured on days 1, 4, 7, 11, 14, 18, and 21 for a regimen where the drugs are administered orally for 21 days in mice xenografted with SK-MEL-30 melanoma cancer cell line.

FIG. 10 is a depiction of in vivo antitumor activity induced by belvarafenib (designated as HM95573) or selumetinib (designated at AZD6244) either alone or their combination administered orally for 17 days in mice xenografted with calu-6 non-small cell lung cancer cell line.

FIG. 11 is a depiction of body weight change induced by belvarafenib (designated as HM95573) or selumetinib (designated at AZD6244) either alone or their combination administered orally for 17 days in mice xenografted with calu-6 non-small cell lung cancer cell line.

### DETAILED DESCRIPTION

In one aspect, the present disclosure is directed to the treatment of NRAS-mutant melanoma cancer by administration of a combination of belvarafenib and cobimetinib.

In one aspect, the present disclosure is directed to the treatment of NRAS-mutant melanoma cancer by administration of a combination of belvarafenib, cobimetinib, and atezolizumab.

### Definitions

As used herein, “NRAS mutant” refers to an NRAS (NRAS proto-oncogene GTPase) oncogene harboring a change (mutation) in the DNA sequence. The NRAS gene encodes the synthesis of the N-Ras protein that is involved in regulating cell division. Without being bound to any particular theory, it is believed that NRAS mutations result in impaired GTPase activity and the locking of NRAS into its activated (GTP-bound) state, independent of upstream RTK activation (see Normanno, N., OncologyPRO, 2015). NRAS mutations in melanomas are associated with aggressive disease and poor prognosis. NRAS mutated predominantly at codon 61 is believed to be implicated in up to 30% of all melanomas. NRAS oncogene mutations are also believed to occur in codons 12 and 13.

As used herein, the terms “patient” and “subject” refer to animals such as mammals, including, but not limited to, primates (e.g., humans), cows, sheep, goats, horses, dogs, cats, rabbits, rats, mice and the like. In certain aspects, the patient or subject is a human.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, the phrase “therapeutically effective amount” refers to an amount of one or more drug compounds that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the overall response rate (ORR). A therapeutically effective amount herein may vary according to factors such as the disease state, age, sex, and weight of the patient, and the ability of the agent to elicit a desired response in the individual. A therapeutically effective amount is also one in which a toxic or detrimental effect of the treatment is outweighed by the therapeutically beneficial effect. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, and enhancing effect of another medication such as via targeting, delaying the progression of the disease, and/or prolonging survival. In the case of a cancer or a tumor, a therapeutically effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. A therapeutically effective amount can be administered in one or more administrations. For purposes of this invention, a therapeutically effective amount of drug, compound, pharmaceutical composition, or pharmaceutical formulation is an amount sufficient to accomplish prophylactic or therapeutic treatment either directly or indirectly. As is understood in the clinical context, a therapeutically effective amount of a drug, compound, or pharmaceutical composition may or may not be achieved in combination with another drug, compound, or pharmaceutical composition. Thus, a therapeutically effective amount may be considered in the context of administering one or more therapeutic agents, and a single agent may be considered to be given in a therapeutically effective amount if, in combination with one or more other agents, a desirable result may be or is achieved.

As used herein, “in combination with” refers to administration of one treatment modality in addition to another treatment modality. As such, “in combination with” refers to administration of one treatment modality before, during, or after administration of the other treatment modality to the individual.

As used herein, the term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject to provide an effective dose of the active ingredient employed.

As used herein, “C” with reference to maximum, minimum, or other metric refers to drug concentration in plasma.

As used herein “area under concentration curve” (AUC) refers to the area under a fitted plasma concentration versus time curve. AUC_{0-∞} refers to area under curve baseline—infinity. AUC_{0-T} is total exposure.

As used herein “inhibit” refers to a decrease in the activity of a target enzyme or other protein, as compared to the activity of that enzyme (or protein) in the absence of the inhibitor. In some aspects, the term “inhibit” means a decrease in activity of at least about 5%, at least about 10%, at least about 20%, at least about 25%, at least about 50%, at least about 60%, at least about 70%, at least about 80%, at least about 90%, or at least about 95%. In other aspects, inhibit means a decrease in activity of about 5% to about 25%, about 25% to about 50%, about 50% to about 75%, or about 75% to about 100%. In some aspects, inhibit means a decrease in activity of about 95% to about 100%, e.g., a decrease in activity of 95%, 96%, 97%, 98%, 99%, or 100%. Such decreases can be measured using a variety of techniques that would be recognizable by one of skill in the art.

As used herein, “progression free survival” (PFS) refers to the time from the treatment of the disease to the first occurrence of disease progression or relapse.

As used herein, “partial response” (PR) refers to at least a 30% decrease in the sum of diameters of target lesions, taking as reference the baseline sum of diameters.

As used herein, “complete response” (CR) refers to disappearance of all target lesions.

As used herein, “progressive disease” (PD) refers to at least a 20% increase in the sum of diameters of target lesions, taking as reference the smallest sum on study (nadir), including baseline and an absolute increase of at least 5 mm. The appearance of one or more new lesions is also considered progression.

As used herein, “overall response rate” (ORR) refers to the rate of a PR or CR occurring after randomization and confirmed ≥28 days later as determined by the investigator using RECIST v1.1.

As used herein, “duration of response” (DOR) refers to the time from the first occurrence of a documented objective response to the time of relapse, as determined by the investigator using RECIST v1.1 or death from any cause during the study, whichever occurs first.

As used herein, the term “RAF inhibitor(s)” refers to a molecule that inhibits at least one of three receptor subtypes (A-RAF, B-RAF, C-RAF) in the MAPK signaling pathway downstream of RAS.

As used herein, the term “MAPK” refers to the mitogen-activated protein kinase pathway or signaling pathway. Also termed the Ras-Raf-MEK-ERK pathway, the MAPK pathway, is a chain or pathway of proteins in the cell that communicates a signal from a receptor on the surface of the cell to the DNA in the nucleus of the cell. In the MAPK pathway, activated RAS activates the protein kinase activity of RAF kinase, RAF kinase phosphorylates and activates MEK (MEK1 and MEK2), MEK phosphorylates and activates a mitogen-activated protein kinase (MAPK) ERK1 and ERK2 (MAPK3 and MAPK1). MAPK phosphorylates ribosomal protein S6 kinase (RPS6KA1; RSK).

As used herein, the term “PD-1 axis inhibitor” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis inhibitor includes a PD-1 inhibitor, a PD-L1 inhibitor, and a PD-L2 inhibitor.

As used herein, the term “PD-1 inhibitor” refers to a molecule that decreases, blocks, inhibits, abrogates or interferes with signal transduction resulting from the interaction of PD-1 with one or more of its binding partners, such as PD-L1 and PD-L2. In some embodiments, the PD-1 inhibitor is a molecule that inhibits the binding of PD-1 to one or more of its binding partners. In a specific aspect, the PD-1 inhibitor inhibits the binding of PD-1 to PD-L1 and/or PD-L2. For example, PD-1 inhibitors include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 inhibitor reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 inhibitor is an anti-PD-1 antibody.

As used herein, the term “PD-L1 inhibitor” refers to a molecule that decreases, blocks, inhibits, abrogates or interferes with signal transduction resulting from the interaction of PD-L1 with either one or more of its binding partners, such as PD-1, B7-1. In some embodiments, a PD-L1 inhibitor is a molecule that inhibits the binding of PD-L1 to its binding partners. In a specific aspect, the PD-L1 inhibitor inhibits binding of PD-L1 to PD-1 and/or B7-1. In some embodiments, the PD-L1 inhibitor include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 inhibitor reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 inhibitor is an anti-PD-L1 antibody.

As used herein, the term “PD-L2 inhibitor” refers to a molecule that decreases, blocks, inhibits, abrogates or interferes with signal transduction resulting from the interaction of PD-L2 with either one or more of its binding partners, such as PD-1. In some embodiments, a PD-L2 inhibitor is a molecule that inhibits the binding of PD-L2 to one or more of its binding partners. In a specific aspect, the PD-L2 inhibitor inhibits binding of PD-L2 to PD-1. In some embodiments, the PD-L2 inhibitor include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 inhibitor reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 inhibitor is an immunoadhesin.

The term “pharmaceutically acceptable salts” denotes salts which are not biologically or otherwise undesirable. Pharmaceutically acceptable salts include both acid and base addition salts. The phrase “pharmaceutically acceptable” indicates that the substance or composition is compatible chemically and/or toxicologically with the other ingredients comprising a formulation, and/or the subject being treated therewith. Acid addition salts are formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid “mesylate”, ethanesulfonic acid, p-toluenesulfonic acid, and salicyclic acid. Base addition salts are formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “detection” includes any means of detecting, including direct and indirect detection.

### Therapeutic Agents

In one aspect, the present disclosure is directed to the combination of belvarafenib or a pharmaceutically acceptable salt thereof and cobimetinib or a pharmaceutically acceptable salt thereof to treat NRAS-mutant melanoma.

In one aspect, the present disclosure is directed to the combination of belvarafenib or a pharmaceutically acceptable salt thereof, cobimetinib or a pharmaceutically acceptable salt, and atezolizumab to treat NRAS-mutant melanoma.

The presently disclosed compounds may be administered in any suitable manner known in the art. In some aspects, the compounds may be administered intravenously, intramuscularly, subcutaneously, topically, orally, transdermally, intraperitoneally, intraorbitally, by implantation, by inhalation, intrathecally, intraventricularly, intratumorally, or intranasally.

It is understood that appropriate doses of the active compound depends upon a number of factors within the knowledge of the ordinarily skilled physician. The dose(s) of the active compound will vary, for example, depending upon the age, body weight, general health, gender, and diet of the subject, the time of administration, the route of administration, the rate of excretion, and any drug combination.

It will also be appreciated that the effective dosage of the compound of the present disclosure, or a pharmaceutically acceptable salts, prodrugs, metabolites, or derivatives thereof used for treatment may increase or decrease over the course of a particular treatment. Changes in dosage may result and become apparent from the results of diagnostic assays.

### Belvarafenib

Belvarafenib is disclosed in PCT application WO 2013/100632, has the chemical name 4-amino-N-(1-((3-chloro-2-fluorophenyl)amino)-6-methylisoquinolin-5-yl)thieno[3,2-d]pyrimidine-7-carboxamide (referred to herein as Formula (I)), and has the following chemical structure:

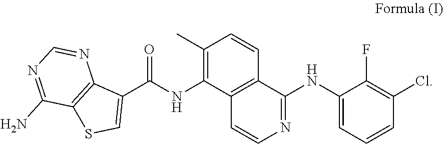 [SMILES: Cc1ccc2c(Nc3cccc(Cl)c3F)nccc2c1NC(=O)c1csc2c(N)ncnc12]

Belvarafenib is a highly potent and selective type II RAF dimer inhibitor (a pan-RAF inhibitor) that provides for selective inhibition of BRAF and CRAF isoforms. In contrast with BRAF^{V600}-selective monomer inhibitors, belvarafenib does not activate the MAPK pathway in non-BRAF V600 mutant cells, but instead sustains the suppression of MAPK signaling by inhibiting BRAF and CRAF dimers, and results in reduced cell proliferation and increased antitumor activity in both BRAF^{V600}- and RAS-mutant tumors.

Belvarafenib inhibits phosphorylation of MEK and ERK in the MAPK pathway in BRAF- or RAS-mutant melanoma, NSCLC, and CRC cell lines. Belvarafenib has been demonstrated to inhibit the growth of BRAF- or RAS-mutant melanoma, NSCLC, CRC, and thyroid cancer cell lines in vitro.

Belvarafenib is a potent and selective inhibitor of the RAF kinases including BRAF V600E mutant (IC₅₀=7 nM), BRAF wild type (IC₅₀=41 nM), and WT RAF-1 (CRAF) (IC₅₀=2 nM), in vitro. Belvarafenib is also capable of inhibiting BRAF/CRAF heterodimers that propagate signaling downstream of activating RAS mutations. When tested in a panel of 189 kinase assays, belvarafenib showed inhibitory activity against 7 other receptor tyrosine kinases (RTKs) (colony stimulating factor 1 receptor (CSF1R), formerly McDonough feline sarcoma (FMS) homolog, discoidin domain receptor tyrosine kinase 1 (DDR1), discoidin domain receptor tyrosine kinase 2 (DDR2), EPHA2, EPHA7, EPHA8, and EPHB2) with >90% inhibition at 1 μM.

Belvarafenib has exhibited significant inhibition of tumor cell growth across a panel of BRAF- or KRAS-mutant non-small cell lung cancer (NSCLC), colorectal cancer (CRC), and thyroid cancer cell lines, as well as NRAS- or BRAF-mutant melanoma cancer cell lines. Belvarafenib has shown inhibitory effects on cell viability in BRAF- and NRAS-mutant melanomas, with median half maximal inhibitory concentrations (IC₅₀s) of 340 nM and 82 nM, respectively. Belvarafenib has exhibited tumor growth inhibition (TGI) in an NRAS-mutant melanoma xenograft model SK-MEL-30 (NRAS^{Q61K} mutant) both as a single agent with maximum TGI of 80% and in combination with cobimetinib with maximum TGI of 89.8%.

Based on experimental evidence of the present disclosure, belvarafenib was found to exhibit a maximum TGI of 130% in a panel of NRAS-mutant melanoma patient-derived xenograft models as follows. A mouse-derived xenograft of patient 1 NRAS^{Q61K} mutant melanoma cells gave a maximum TGI of 85%. A mouse-derived xenograft of patient 2 NRAS^{Q61R} mutant melanoma cells gave a maximum TGI of 107%. A mouse-derived xenograft of patient 3 NRAS^{G12C} mutant melanoma cells gave a maximum TGI of 90%. A mouse-derived xenograft of patient 4 NRAS^{Q61R} mutant melanoma cells gave a maximum TGI of 127%. A mouse-derived xenograft of patient 5 NRAS^{Q61R} mutant melanoma cells gave a maximum TGI of 130%. A mouse-derived xenograft of patient 6 NRAS^{Q61K} mutant melanoma cells gave a maximum TGI of 110%. A mouse-derived xenograft of patient 7 NRAS^{Q61R} mutant melanoma cells gave a maximum TGI of 1²⁴%. A mouse-derived xenograft of patient 8 NRAS^{Q61R} mutant melanoma cells gave a maximum TGI of 115%. A mouse-derived xenograft of patient 9 NRAS^{Q61K} mutant melanoma cells gave a maximum TGI of 125%.

The in vitro antitumor effects of belvarafenib have translated into efficacy in various mouse xenograft models. Belvarafenib shows dose-dependent inhibition of tumor growth in mouse xenograft models as a monotherapy against BRAF- and NRAS-mutant melanoma, against KRAS mutant non-small cell lung cancer (NSCLC), and against BRAF mutant colorectal cancer (CRC) mouse xenograft models.

Belvarafenib has been shown in clinical trials to provide safe and efficacious therapy against a number of cancers. For instance, a completed, open-label, Phase Ia, dose-escalation investigated several doses and schedules of belvarafenib in patients with solid tumors harboring mutations in BRAF, KRAS, or NRAS genes (clinical trial NCT02405065). Efficacy was analyzed for 67 of 72 subjects who had at least 1 post-baseline tumor assessment. Best overall response rate (BORR) was 8.96% (6/67 subjects), objective response rate (ORR) was 4.48% (3/67 subjects) with partial response (PR) as confirmed best overall response (2 subjects with melanoma and a subject with gastrointestinal stromal tumor). Disease control was observed in 50.57% (34/67) of subjects treated with belvarafenib 100 mg QD dose level or above. Fifty-nine (88.06%) subjects developed an event (progression of disease or death), all of which were reported as progressive disease (PD). In addition, median progression-free survival was 11.53 weeks and the 95% confidence interval for the median was [7.12 weeks, 13.38 weeks). In updated results, BORR was 10.45% (7/67 subjects) and the 95% exact confidence interval was [4.30%, 20.35%]; ORR remains at 4.48% (3/67 subjects). In addition, subgroup re-analysis for BRAF-mutant melanoma subjects showed 7.69% (1/13 subjects) of BORR, DCR, median PFS, and time to progression were not changed in total subjects. Median DOR was elevated to 30.18 weeks in 800 mg BID group and 23.99 weeks in total group including DOR of a subject which is 100.29 weeks.

In another open-label, Phase Ib, dose-expansion study, belvarafenib was evaluated at a dose of 450 mg BID in patients with solid tumors harboring mutations in BRAF, KRAS, or NRAS genes. Efficacy was analyzed for 59 of 63 subjects who had at least 1 dose of belvarafenib after enrollment and had at least 1 post-baseline tumor assessment. BORR was 11.86% (7/59 subjects), ORR was 6.78% (4/59 subjects) with PR as confirmed best overall response (3 subjects with melanoma and a subject with CRC). Disease control was observed in 35.59% (21/59) of subjects. Fifty (84.75%) of 59 subjects developed an event (progression of disease or death), all of which were reported as PD except 1 death case. In addition, median progression-free survival (PFS) was 7.83 weeks and the 95% confidence interval for the median was [7.26 weeks, 8.26 weeks]. Median duration of response (DOR) of a total response in this study was 15.66 weeks from 7 responders. Among them, 2 BRAF-mutant melanoma responders showed 22.49 weeks of median DOR.

In another Phase I, single dose, randomized, crossover relative bioavailability and food effect study in healthy subjects, the influence of a formulation change from the Phase I to Phase II tablet on belvarafenib exposure was evaluated (clinical trial GP41348). A total of 18 healthy subjects were enrolled in the study and received the following randomized treatments: one 150-mg and one 50-mg Phase I tablet in a fed state, two 100-mg Phase II tablets in a fed state, or two 100-mg Phase II tablets in a fasted state, with a 18-day washout between treatments. There was a positive effect of food on belvarafenib exposure in the fed state compared to the fasted state. Belvarafenib exposure, Cₘₐₓ and AUC_{0-inf}, were increased by approximately 2.2- and 2.8-fold, respectively, when belvarafenib was administered in the fed state compared to the fasted state in healthy subjects at a 200 mg single dose. No serious adverse events, adverse events of special interest, or deaths were reported in the study.

Belvarafenib, or a pharmaceutically acceptable salt thereof, is suitably dosed at from about 200 mg to about 1300 mg, from about 400 mg to about 1200 mg, from about 600 mg to about 1200 mg, or from about 800 mg to about 1000 mg, per day on an active ingredient basis. In some such aspects, the subject is administered about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, or about 1300 mg of belvarafenib, or a pharmaceutically acceptable salt thereof, per day. In some other such aspects, the subject is administered about 250 mg, about 300 mg, about 350 mg, about 400 mg, about 450 mg, or about 500 mg of belvarafenib, or a pharmaceutically acceptable salt thereof, twice per day (“BID”). In some other such aspects, the subject is administered about 300 mg or about 400 mg of belvarafenib or a pharmaceutically acceptable salt thereof twice per day. Other dosing regimens may be used to achieve a total daily dose, such as three doses per day or four doses per day. In any such dosing regimen, such as two, three or four times per day, each dose may suitably be about equal. For instance, if the daily dose is 900 mg, two daily doses of 450 mg each or three daily doses of 300 mg each could be used.

In some aspects, belvarafenib may be dosed on days 1 to 21 of a 28-day cycle. In some aspects, belvarafenib may be dosed on days 1 to 28 of a 28-day cycle.

### Cobimetinib

Cobimetinib has the chemical name (S)-[3,4-Difluoro-2-(2-fluoro-4-iodophenylamino)phenyl][3-hydroxy-3-(piperidin-2-yl)azetidin-1-yl]methanone, and having the below structure (II):

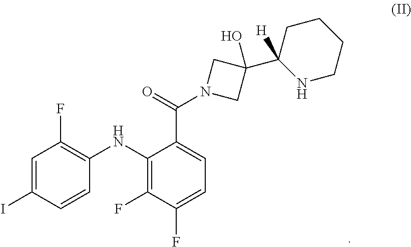 [SMILES: O=C(c1ccc(F)c(F)c1Nc1ccc(I)cc1F)N1CC(O)([C@@H]2CCCCN2)C1]

Cotellic® is the fumarate salt of cobimetinib. Cobimetinib is described in U.S. Pat. Nos. 7,803,839 and 8,362,002, each of which is incorporated by reference in its entirety. Cobimetinib is a reversible, potent, and highly selective inhibitor of MEK1 and MEK2 (central components of the RAS/RAF/MEK/ERK (MAPK)) pathway and has single agent anti-tumor activity in multiple human cancer models.

Cobimetinib is a potent and highly selective inhibitor of MEK1 and MEK2, central components of the MAPK pathway signaling downstream through ERK to promote normal cell growth. Upregulation of the pathway leads to constitutive signaling and malignant transformation, and occurs in a large fraction of tumors, frequently owing to oncogenic activating mutations in RAS and BRAF (Bamford S, Dawson E, Forbes S, et al. The COSMIC (Catalogue of Somatic Mutations in Cancer) database and website. Br J Cancer 2004; 91:355-8). Cancer cells transformed by BRAF^{V600} are exceptionally sensitive to MEK inhibition in vitro. Allosteric MEK inhibitors can result in G1 phase growth arrest in melanoma cells (Solit D B, Garraway L, Pratilas C, et al. BRAF mutation predicts sensitivity to MEK inhibition. Nature 2006, 439:358-62; Haass N K, Sproesser K, Nguyen T K, et al. The mitogen-activated protein extracellular signal-regulated kinase inhibitor AZD6244 (ARRY-142886) induces growth arrest in melanoma cells and tumor regression when combined with docetaxel. Clin Cancer Res 2008, 14:230-9).

In vitro, MEK inhibitors reduce cell proliferation, soft agar colony formation, and matrigel invasion of BRAF^{V600} mutation-positive melanoma cells and are also effective against BRAF^{V600} mutation-positive melanoma xenografts (Solit et al. 2006). NRAS-mutant melanomas are highly dependent on MAPK pathway signaling, and MAPK signaling is required for tumor progression in NRAS-mutant melanoma mouse models (Dorard C, Estrada C, Barbotin C, et al. RAFproteins exert both specific and compensatory functions during tumour progression of NRAS-driven melanoma. Nat Commun 2017; 8:1-13). In nonclinical studies, CI-1040 (MEK inhibitor) decreased cell viability and pathway signaling in SK-MEL-130 (NRAS^{Q61R} mutant) melanoma cells (Solit et al. 2006), and binimetinib (MEK inhibitor) inhibited cell growth in BRAF- and NRAS-mutant melanoma cell lines (Winski L, Anderson D, Bouhana K, et al. MEK162 (ARRY-162), a novelMEK 1/2 inhibitor, inhibits tumor growth regardless of KRas Raf pathway mutations. EORTC-NCI-AACR, Berlin [poster]. 2010).

The pharmacokinetics (PK) of cobimetinib administered as a single agent have been characterized in cancer patients following oral administration after single and multiple dosing in the Phase Ia dose escalation Study MEK4592g which included evaluation of a cobimetinib dose of 60 mg per day in patients who harbored a BRAF, NRAS, or KRAS mutation. Overall 6 patients (all of whom had melanoma; 6.2%) had a confirmed partial response (PR), 28 patients (28.9%) had stable disease (SD), and 40 patients (41.2%) had progressive disease. Out of the 14 colorectal cancer (CRC) patients, all patients experienced progressive disease (PD). In Stage III of Study MEK4592g, 18 patients were accrued, and best overall response was assessed for 14 of 18 patients. Four patients (22.2%) had SD as their best overall response, and 2 patients (11.1%) had unconfirmed tumor responses.

Cobimetinib has a moderate rate of absorption (median time to maximum concentration [tₘₐₓ] of 1 to 3 hours) and a mean terminal half-life (t½) of 48.8 hours (a range of 23.1 to 80 hours). Cobimetinib binds to plasma proteins (95%) in a concentration-independent manner. Cobimetinib exhibits linear pharmacokinetics in the dose range of 0.05 mg/kg (approximately 3.5 mg/kg for 70 kg adult) to 80 mg and the absolute bioavailability was determined to be 45.9% (90% CI: 39.74%, 53.06%) in study MEK4952g in healthy subjects. Cobimetinib pharmacokinetics are not altered when administered in the fed state compared with administration in the fasted state in healthy subjects. Since food does not alter cobimetinib pharmacokinetics, cobimetinib can be administered with or without food. The proton pump inhibitor rabeprazole appears to have a minimal effect on cobimetinib pharmacokinetics, whether administered in the presence or absence of a high-fat meal compared with cobimetinib administration alone in the fasted state. Thus, increase in gastric pH does not affect cobimetinib pharmacokinetics, indicating it is not sensitive to alterations in gastric pH.

Cobimetinib salts, crystalline forms and prodrugs are within the scope of the present disclosure. Cobimetinib, preparative methods, and therapeutic uses are disclosed in International Publication Numbers WO 2007/044515, WO 2014/027056 and WO 2014/059422, each of which is incorporated herein by reference in its entirety. For instance, in some aspects of the present disclosure, the MEK inhibitor is crystalline hemifumarate cobimetinib polymorph Form A.

Cobimetinib, or a pharmaceutically acceptable salt thereof, doses within the scope of the present disclosure are from about 20 mg to about 100 mg, from about 40 mg to about 80 mg, or about 60 mg per day on an active ingredient basis. In some aspects, the cobimetinib dose is about 60 mg, about 40 mg, or about 20 mg.

Cobimetinib is suitably administered once daily. In some aspects, Cobimetinib is administered once daily for 21 consecutive days of a 28-day treatment cycle. In some aspects, cobimetinib is administered once daily on days 1 to 21 of a 28-day treatment cycle. In some aspects, cobimetinib is administered once daily on days 3 to 23 of a 28-day treatment cycle.

### Atezolizumab

Atezolizumab is also known as MPDL3280A (CAS Registry Number: 1380723-44-3). Atezolizumab is a humanized IgG1 monoclonal antibody that targets PD-L1 and inhibits the interaction between PD-L1 and its receptors, PD-1 and B7-1 (also known as CD80), both of which function as inhibitory receptors expressed on T cells. Therapeutic blockade of PD-L1 binding by atezolizumab has been shown to enhance the magnitude and quality of tumor specific T cell responses, resulting in improved anti tumor activity (Fehrenbacher L, Spira A, Ballinger M, et al. Atezolizumab versus docetaxelfor patients with previously treated non-small-cell lung cancer (POPLAR): a multicentre, open label, phase 2 randomised controlled trial. Lancet 2016, 387:1837-46; Rosenberg J E, Hoffman-Censits J, Powles T, et al. Atezolizumab inpatients with locally advanced and metastatic urothelial carcinoma who have progressed following treatment with platinum-based chemotherapy: a single-arm, multicentre, phase 2 trial. Lancet 2016, 387:1909-20). Atezolizumab has minimal binding to Fc receptors, thus eliminating detectable Fc effector function and associated antibody-mediated clearance of activated effector T cells.

The pharmacokinetics of atezolizumab administered as a single agent have been characterized based on clinical data from study PCD4989g and are consistent with a currently ongoing Phase III Study WO29522 in first line treatment of TNBC. Atezolizumab anti-tumor activity has been observed across doses from 1 to 20 mg/kg. Overall, atezolizumab exhibits pharmacokinetics that are both linear and consistent with typical IgG1 antibodies for doses ≥1 mg/kg every three weeks (q3w). Pharmacokinetic data (Bai S, Jorga K, Xin Y, et al., A guide to rational dosing of monoclonal antibodies, Clin Pharmacokinet 2012; 51:119-35, incorporated by reference herein in its entirety) does not suggest any clinically meaningful differences in exposure following a fixed dose or a dose adjusted for weight. Atezolizumab dosing schedules of q3w and q2w have been tested. A fixed dose of atezolizumab 800 mg every two weeks (q2w) (equivalent to a body weight-based dose of 10 mg/kg q2w) results in equivalent exposure to the Phase III dose of 1200 mg administered every three weeks (q3w). The q3w schedule is being used in multiple Phase III studies of atezolizumab monotherapy across multiple tumor types and the q2w predominantly used in combination with chemotherapy regimens. In Study PCD4989g, the Kaplan-Meier estimated overall 24-week progression-free survival (PFS) rate was 33% (95% CI: 12%, 53%).

The atezolizumab dose of the present disclosure is suitably from about 500 mg to about 2000 mg, from about 500 mg to about 1000 mg, from about 750 mg to about 1000 mg, from about 750 mg to about 2000 mg, from about 1000 mg to about 2000 mg, from about 1500 mg to about 1750 mg. In some such aspects, the subject is administered about 840 mg of atezolizumab. In some such aspects, the subject is administered about 1680 mg of atezolizumab. In some aspects, the subject is administered atezolizumab every 14 days of a 28-day treatment cycle. In some other aspects, the subject is administered atezolizumab on days 1 and 15 of the 28-day treatment cycle. In other aspects, the subject is administered atezolizumab every four weeks of the 28-day treatment cycle. In other aspects, the subject is administered atezolizumab on day one of the 28-day treatment cycle. In one aspect, the subject is administered about 1680 mg of the atezolizumab on day one of the 28-day treatment cycle.

### Melanoma

Melanoma within the scope of the present disclosure carries an NRAS mutation. In some aspects, the melanoma is advanced as defined by the American Joint Committee on Cancer, 8th revised edition (Amin M B, Edge S, Greene F, et al. (Eds.). AJCC Cancer Staging Manual (8th edition). Springer International Publishing: American Joint Commission on Cancer; 2017), harboring an NRAS-activating mutation. Determination of NRAS mutation-positive status may be made through use of a clinical mutation test approved by a national health authority, such as, for instance and without limitation, the U.S. Food and Drug Administration, the College of American Pathologists, or CE-marked (European conformity) in vitro diagnostic in E.U. countries. In some aspects, NRAS mutation-positive status is defined as a mutation occurring in NRAS gene codons 12, 13 ofexon 2, and codon 61 of exon 3.

In some aspects, the melanoma carries a NRAS^{Q61K} mutation, a NRAS^{Q61R} mutation, a NRAS^{G12C} mutation, NRAS^{Q61L} mutation, a NRAS^{G13D} mutation, or any combination thereof. In some other aspects, the melanoma carries a NRAS^{G12D} mutation, a NRAS^{G12C} mutation, or a combination thereof

In some aspects, the melanoma is metastatic or unresectable.

In some aspects, the melanoma continued to progress in a subject after treatment with immunotherapy, BRAF V600E therapy, or a combination of immunotherapy and BRAF V600E therapy.

In some aspects, the melanoma continue to progress in a subject who was previously administered a course of treatment with an anti-PD-1 drug or anti-PD-L1 drug.

### Combination Therapies

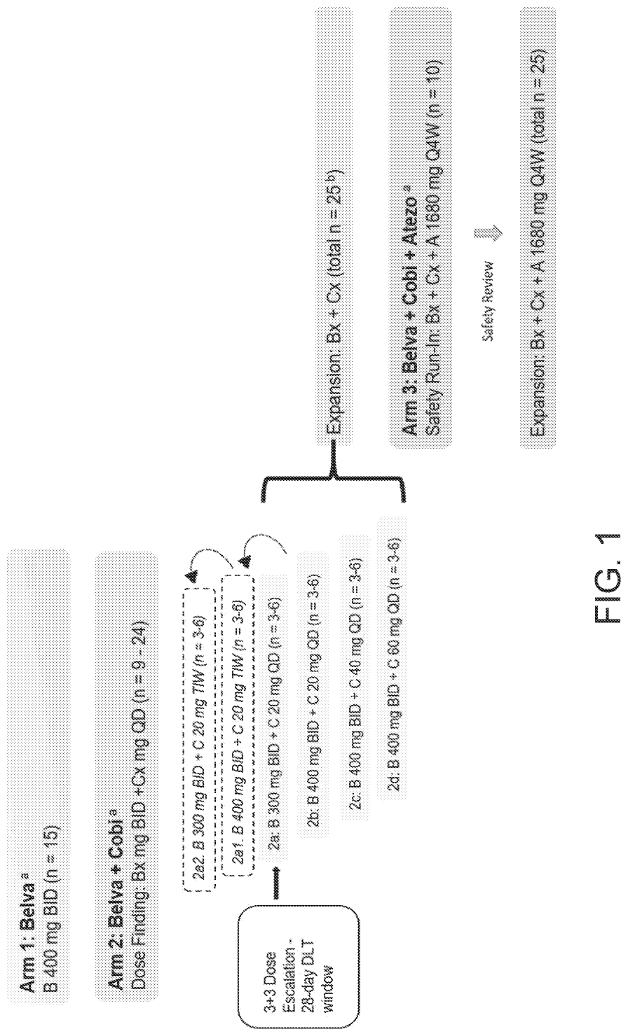

The combination therapies of (i) belvarafenib and cobimetinib and (ii) belvarafenib, cobimetinib and atezolizumab will be evaluated according to the protocol depicted in FIG. 1 and described herein.

The protocol is directed to a Phase Ib, open-label, multicenter study to evaluate the safety, pharmacokinetics, and preliminary anti-tumor activity of belvarafenib as a single agent and in combination with either cobimetinib or cobimetinib plus atezolizumab in patients with NRAS-mutant metastatic or unresectable locally advanced cutaneous melanoma who have received up to two lines of systemic anti-cancer therapy that included anti-PD-1/PD-L1 therapy. Patients may have been treated with anti-PD-1 or anti-PD-L1 in the adjuvant setting.

The study will evaluate three treatment regimens in three respective arms: a belvarafenib monotherapy arm (Belva arm) of up to 15 patients; a belvarafenib plus cobimetinib arm of up to approximately 43 patients enrolled in an initial dose-finding phase (from 9 to 24 patients) followed by an expansion phase of the selected dose with up to 25 patients; and a belvarafenib plus cobimetinib plus atezolizumab arm of approximately 25 total patients, with a safety run-in phase (10 patients) followed by an expansion phase.

In some aspects, belvarafenib is administered with food.

In some aspects, the combination therapies of the present disclosure are characterized by the absence of the development of squamous cell carcinoma in human subjects.

### Belvarafenib Monotherapy Arm

Up to 15 patients will be enrolled in the Belva arm and will receive 400 mg belvarafenib BID in tablet form on Days 1-28 of each 28-day cycle.

Plasma samples for the PK characterization of belvarafenib will be collected as outlined in Table 1. The sampling schedule for all three arms is designed to enable characterization of belvarafenib pharmacokinetics using non-compartmental analysis and/or population PK (popPK) methodology. Belvarafenib PK data from the combination arms (where belvarafenib is co-administered with either cobimetinib or cobimetinib plus atezolizumab) will be compared with single-agent belvarafenib in the belvarafenib monotherapy arm or previous Phase I studies to evaluate if belvarafenib exposures are altered. In Table 1: “C” refers to cycle; “D” refers to day; “PBMC” refers to peripheral blood mononuclear cell; and “PK” refers to pharmacokinetic.

TABLE 1
Schedule of Pharmacokinetic, Immunogenicity, and Biomarker
Samples for Belvarafenib monotherapy arm

| Visit | Timepoint | Sample Type | ECG Collection |
|---|---|---|---|
| C1D1 | Predose | Belvarafenib PK (plasma) | Matched triplicate |
| | | Biomarker (Blood for WGS, | 12-lead ECG |
| | | plasma and PBMC) | |
| | 2 hours | Belvarafenib PK (plasma) | Matched triplicate |
| | postdose | | 12-lead ECG |
| | 4 hours | Belvarafenib PK (plasma) | Matched triplicate |
| | postdose | | 12-lead ECG |
| | 6 hours | Belvarafenib PK (plasma) | Matched triplicate |
| | postdose | | 12-lead ECG |
| C2D1 | Predose | Belvarafenib PK (plasma) | Matched triplicate |
| | | Biomarker (Blood for | 12-lead ECG |
| | | plasma and PBMC) | |
| | 2 hours | Belvarafenib PK (plasma) | Matched triplicate |
| | postdose | | 12-lead ECG |
| | 4 hours | Belvarafenib PK (plasma) | Matched triplicate |
| | postdose | | 12-lead ECG |
| | 6 hours | Belvarafenib PK (plasma) | Matched triplicate |
| | postdose | | 12-lead ECG |
| C3D1 | Predose | Belvarafenib PK (plasma) | Matched triplicate |
| | | | 12-lead ECG |
| Cycles 4, 8, | Predose | Biomarker (Blood for | |
| and 16, Day 1 | | plasma and PBMC) | |

| Treatment discontinuation visit | Belvarafenib PK (plasma) | Matched triplicate |
|---|---|---|
| (≤30 days after final dose) | Biomarker (Blood for | 12-lead ECG |
| | plasma and PBMC) | |

### Belvarafenib and Cobimetinib Arm

Patients will be enrolled in the Belva+Cobi arm in two phases: an initial dose-finding phase following by an expansion phase.

It is believed that the combination of belvarafenib with cobimetinib will result in deeper suppression of ERK output and in greater TGI in tumors that exhibit MAPK pathway dependency (e.g., KRAS, NRAS, or BRAF mutations), compared to single-agent MEK inhibitor or RAF inhibitor. If is further believed that synergy between RAF and MILK inhibitors may be attributed to increased dependency of RAS-mutant tumors on RAF kinase signaling in the presence of a MEK inhibitor.

The purpose of the dose-finding phase will be to identify the recommended dose for belvarafenib and cobimetinib when used in combination.

Approximately 9 to 24 patients will be enrolled in this phase. Dosing groups of 3 to 6 patients each will be treated in accordance with the treatment regimens and will receive belvarafenib at 300 or 400 mg BID on Days 1-28 of each 28-day cycle in combination with cobimetinib at 20, 40, or 60 mg QD on Days 1-21 of each treatment cycle. The dose-finding phase will consist of a traditional 3×3 schema, with a 28-day dose limiting toxicity (DLT) assessment window (1 cycle). As belvarafenib is a potent RAF inhibitor and cobimetinib is a potent MEK inhibitor, dose finding with DLT criteria in the belvarafenib+cobimetinib arm will be implemented to characterize potential compounded effects of MAPK pathway inhibition.

DLTs are defined as any one of the following adverse events determined by the investigator to probably be related to belvarafenib and/or cobimetinib, irrespective of outcome, unless such events are attributed by the investigator to another clearly identifiable cause (e.g., documented disease progression, concomitant or preexisting medication, or intercurrent illness). The events are as follows. Grade ≥3 nausea, vomiting, or diarrhea despite maximal supportive medications lasting for ≥3 days. Grade ≥3 hemorrhage. Grade ≥2 visual disorders (limiting instrumental activities of daily living) that do not resolve to baseline within 14 days. Grade ≥3 left ventricular ejection fraction (LVEF) decline, defined as a resting LVEF of 39%-20% or an LVEF of 40%-49% and ≥10-point drop from baseline. Grade ≥4 anemia. Grade 4 neutropenia or thrombocytopenia lasting >7 days. Grade 3 thrombocytopenia with clinically significant bleeding. Severe hepatotoxicity defined as: Grade ≥3 elevation of total bilirubin or ALT/AST or ALP lasting >7 days, with the following exception: for patients with Grade 2 hepatic transaminase or ALP at baseline as a result of metastases, hepatic transaminase or ALP ≥10×ULN will be considered a DLT; Any increase in hepatic transaminase (ALT or AST) >3×baseline in combination with either an increase in direct bilirubin >2×upper limit of normal (ULN) or clinical jaundice, in the absence of cholestasis or other contributory factors (e.g., worsening of metastatic disease, concomitant exposure to known hepatotoxic agent, or documented infectious etiology) (This is suggestive of potential drug-induced liver injury (according to Hy's Law)). QTc interval corrected using Fridericia's method (QTcF) increased >60 ms compared to baseline (predose) and/or absolute QTcF values >500 ms (confirmed by repeat measurement). Other Grade ≥3 non-hematologic/non-hepatic major organ adverse event, excluding the following: Grade 3 rash that resolves to Grade ≤2 within 7 days with appropriate supportive care; Grade ≥3 fatigue that resolves to Grade ≤2 within 7 days; Grade 3 fever (as defined by >40° C.) for ≤24 hours; Grade 3 laboratory abnormality that is asymptomatic and deemed by the investigator not to be clinically significant; and alopecia of any grade. Any death not clearly due to the underlying disease or extraneous cause.

In the dose-finding phase for the combination of belvarafenib and cobimetinib, the starting dose of belvarafenib will be 300 mg PO BID in the first cohort. The belvarafenib dose will be increased to 400 mg BID in the subsequent cohorts. The starting dose of cobimetinib in combination with belvarafenib will be 20 mg daily for first 21 days of each 28-day cycle in the first two cohorts of the dose-finding phase. The cobimetinib dose will be increased by 20-mg increments up to a maximum dose of 60 mg daily for first 21 days of each 28-day cycle or until a safety threshold is observed. After belvarafenib and cobimetinib doses have been selected in the dose-finding phase, those doses will be further evaluated in up to 25 patients enrolled in the belvarafenib+cobimetinib arm during the expansion phase.

Dose finding will occur in accordance with the following rules irrespective of the duration of the DLT window. A minimum of 3 patients will initially be enrolled in each dose-finding cohort. If none of the first 3 DLT-evaluable patients experiences a DLT, enrollment of the next cohort at the next highest dose level may proceed. If 1 of the first 3 DLT-evaluable patients experiences a DLT, the cohort will be expanded to a minimum of 6 patients. If there are no further DLTs in the first 6 DLT evaluable patients, enrollment of the next cohort at the next highest dose level may proceed. If 2 or more of the first 6 DLT-evaluable patients in a cohort experience a DLT, the MTD will have been exceeded and dose escalation will stop. The preceding cohort will also be expanded to a minimum of 6 patients, unless 6 patients have already been evaluated at that dose level. If the MTD is exceeded at any dose level, the highest dose at which fewer than 2 of first 6 DLT-evaluable patients (i.e., <33%) experience a DLT will be declared the MTD. If the MTD is not exceeded at any dose level, the highest combination of belvarafenib and cobimetinib doses administered in this study will be declared the maximum administered dose (MAD). Any dose level may be expanded in the absence of a DLT if warranted based on evaluation of non-DLT adverse events by the Sponsor and investigator.

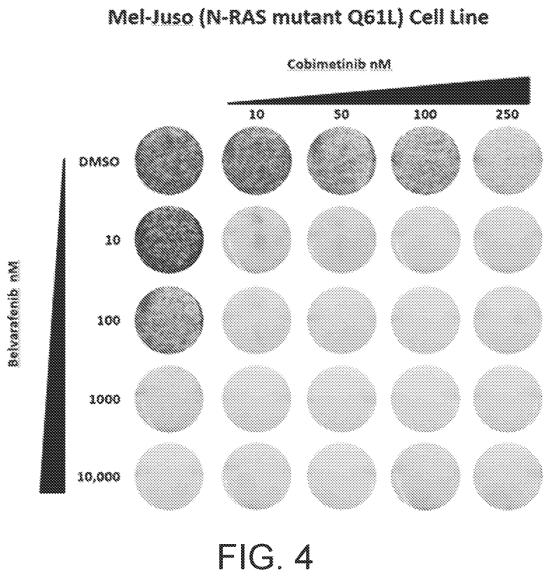

If two DLTs are present in the first 6 patients in the first dose-finding cohort (belvarafenib 300 mg BID plus cobimetinib 20 mg QD daily for first 21 days of each 28-day cycle), de-escalation will occur for cobimetinib from QD dosing to dosing TIW. A de-escalation cohort will open for enrollment, with an increased dose of belvarafenib at 400 mg BID and the dose reduction of cobimetinib to 20 mg TIW for the first 21 days out of each 28-day cycle (see FIG. 4).

If two DLTs are present in the first 6 patients of the de-escalation cohort (belvarafenib 400 mg BID plus cobimetinib 20 mg TIW), dose reduction of belvarafenib will be permitted in a new cohort at belvarafenib 300 mg BID plus cobimetinib 20 mg TIW for first 21 days out of each 28-day cycle. No further dose-reduced cohorts will be opened for consideration beyond that point.

Any patient in the dose-finding phase who does not complete the DLT assessment window of 28 days for a reason other than a DLT will be considered non-evaluable for DLT assessment and may be replaced by an additional patient at the same dose level. Additionally, patients who do not experience a DLT but miss >14 doses of belvarafenib, >5 doses of cobimetinib on the 20 mg QD schedule, and/or >doses of cobimetinib on the 20 mg TIW schedule, regardless of consecutiveness of the missed doses, will be considered non-evaluable for DLT assessment and may be replaced by an additional patient at the same dose level. The dose and/or frequency of belvarafenib and/or cobimetinib may be reduced during the DLT assessment window for management of DLTs with consultation with the Medical Monitor. Patients who do not experience a DLT and receive supportive care during the DLT assessment window that confounds the evaluation of DLTs may be considered non-evaluable for DLT assessment and replaced at the discretion of the Medical Monitor.

Plasma samples for the PK characterization of cobimetinib will be collected as outlined in Tables 2 and 3. The sampling schedule for dose finding in the belvarafenib+cobimetinib arm is designed to enable characterization of cobimetinib pharmacokinetics using non-compartmental analysis and/or popPK methodology. The sampling schedule will move to a sparse sampling schedule in the belvarafenib+cobimetinib expansion and the belvarafenib+cobimetinib+atezolizumab arm and is designed to enable estimation of key cobimetinib PK parameters using popPK methodology. Cobimetinib PK data from the belvarafenib+cobimetinib arm and the belvarafenib+cobimetinib+atezolizumab arm will be compared with single-agent cobimetinib to evaluate if cobimetinib exposures are altered. In Tables 2 and 3: “C” refers to cycle; “D” refers to day; “PBMC” refers to peripheral blood mononuclear cell; and “PK” refers to pharmacokinetic.

TABLE 2
Belvarafenib + Cobimetinib Arm (Arm 2), Dose-Finding Cohorts

| Visit | Timepoint | Sample Type | ECG Collection |
|---|---|---|---|
| C1D1 | Predose | Belvarafenib PK (plasma) | Matched triplicate |
| | | Cobimetinib PK (plasma) | 12-lead ECG |
| | | Biomarker (Blood for | |
| | | WGS, plasma, and PBMC) | |
| | 2 hours | Belvarafenib PK (plasma) | Matched triplicate |
| | postdose | Cobimetinib PK (plasma) | 12-lead ECG |
| | 4 hours | Belvarafenib PK (plasma) | Matched triplicate |
| | postdose | Cobimetinib PK (plasma) | 12-lead ECG |
| | 6 hours | Belvarafenib PK (plasma) | Matched triplicate |
| | postdose | Cobimetinib PK (plasma) | 12-lead ECG |
| C1D15 | Predose | Belvarafenib PK (plasma) | Matched triplicate |
| | | Cobimetinib PK (plasma) | 12-lead ECG |
| | 2 hours | Belvarafenib PK (plasma) | Matched triplicate |
| | postdose | Cobimetinib PK (plasma) | 12-lead ECG |
| | 4 hours | Belvarafenib PK (plasma) | Matched triplicate |
| | postdose | Cobimetinib PK (plasma) | 12-lead ECG |
| | 6 hours | Belvarafenib PK (plasma) | Matched triplicate |
| | postdose | Cobimetinib PK (plasma) | 12-lead ECG |
| C2D1 | Predose | Belvarafenib PK (plasma) | Matched triplicate |
| | | Cobimetinib PK (plasma) | 12-lead ECG |
| | | Biomarker (Blood for | |
| | | plasma and PBMC) | |
| C3D1 | Predose | Belvarafenib PK (plasma) | Matched triplicated |
| | | Cobimetinib PK (plasma) | 12-lead ECG |
| Cycles 4, 8, | Predose | Biomarker (Blood for | |
| and 16, Day 1 | | plasma and PBMC) | |

| | | |
|---|---|---|
| Treatment | Belvarafenib PK (plasma) | Matched triplicated |
| discontinuation | Cobimetinib PK (plasma) | 12-lead ECG |
| visit (≤ 30 days | Biomarker (Blood for | |
| after final dose) | plasma and PBMC) | |

TABLE 3
Belvarafenib + Cobimetinib Arm (Arm 2), expansion Phase

| Visit | Timepoint | Sample Type | ECG Collection |
|---|---|---|---|
| C1D1 | Predose | Belvarafenib PK (plasma) | Matched triplicate |
| | | Cobimetinib PK (plasma) | 12-lead ECG |
| | | Biomarker (Blood for | |
| | | WGS, plasma, and PBMC) | |
| | 2-4 hours | Belvarafenib PK (plasma) | Matched triplicate |
| | postdose | Cobimetinib PK (plasma) | 12-lead ECG |
| C2D1 | Predose | Biomarker (Blood for | |
| | | plasma and PBMC) | |
| C2D15 | Predose | Belvarafenib PK (plasma) | Matched triplicate |
| | | Cobimetinib PK (plasma) | 12-lead ECG |
| | 2-4 hours | Belvarafenib PK (plasma) | Matched triplicate |
| | postdose | Cobimetinib PK (plasma) | 12-lead ECG |
| Cycles 4, 8, | Predose | Biomarker (Blood for | |
| and 16, Day 1 | | plasma and PBMC) | |

| Treatment discontinuation visit | Belvarafenib PK (plasma) | Matched triplicate |
|---|---|---|
| (≤ 30 days after final dose) | Cobimetinib PK (plasma) | 12-lead ECG |
| | Biomarker (Blood for | |
| | plasma and PBMC) | |

### Belvarafenib, Cobimetinib, and Atezolizumab Arm

Patients will be enrolled in the belvarafenib+cobimetinib+atezolizumab arm in two phases: an initial safety run-in phase followed by an expansion phase.

It is believed that the combination of belvarafenib, cobimetinib, and atezolizumab will provide for increased anti-tumor activity compared with monotherapy. More particularly, it is believed that the combination of belvarafenib and atezolizumab will provide for concurrent inhibition of RAF isoforms and the PD-1 pathway resulting in improved efficacy in those tumors carrying mutations in RAS and RAF.

Approximately 10 patients will be enrolled in the safety run-in phase after the recommended doses for belvarafenib and cobimetinib have been determined. The arm will only be activated after the dose determinations have been made and an assessment of the corresponding safety data from the belvarafenib+cobimetinib dose finding group has been completed, with all relevant data reviewed.

After all patients in the safety run-in phase have been followed for 28 days, the Sponsor Safety Committee will review the safety data to determine if the expansion phase should open. If <80% of the patients in the safety run-in phase experience a Grade ≥3 adverse event during the 28-day safety run-in period, then enrollment in the expansion phase will begin.

While the expansion phase is enrolling, the Sponsor Safety Committee will reassess the safety data after the initial 10 patients have received at least 90 days of study treatment, which will allow for thorough evaluation of toxicity, including time course, late onset events, and reversibility. The expansion phase will enroll up to 25 patients.

In each 28-day cycle, patients will receive belvarafenib at the recommended dose BID on Days 1-28, cobimetinib at the recommended dose on Days 1-21, and atezolizumab 1680 mg by IV infusion every 4 weeks (Q4W).

Serum samples for the PK characterization of atezolizumab will be collected as outlined in Table 4. The sampling schedule in the belvarafenib+cobimetinib +atezolizumab arm is designed to enable estimation of key atezolizumab PK parameters using popPK methodology. Atezolizumab PK data from the belvarafenib+cobimetinib+atezolizumab arm will be compared with single-agent atezolizumab to evaluate if atezolizumab exposures are altered In Table 4: “C” refers to cycle; “D” refers to day; “PBMC” refers to peripheral blood mononuclear cell; and “PK” refers to pharmacokinetic. It is believed that the sample schedule for atezolizumab anti-drug antibody (ADA) assessments should allow for evaluation of the immunogenicity of atezolizumab given on a Q4W schedule when administered in combination with belvarafenib and cobimetinib.

TABLE 4
Belvarafenib + Cobimetinib + Atezolizumab Arm
(Arm 3), Safety Run-In and Expansion Phases

| Visit | Timepoint | Sample Type | ECG Collection |
|---|---|---|---|
| C1D1 | Predose | Belvarafenib PK (plasma) | Matched triplicate |
| | | Cobimetinib PK (plasma) | 12-lead ECG |
| | | Biomarker (Blood for | |
| | | WGS, plasma, and PBMC) | |
| | Prior to the | Atezolizumab PK (serum) | |
| | first infusion | Atezolizumab ADA (serum) | |
| | 30 ± 10 | Atezolizumab PK (serum) | |
| | minutes post- | | |
| | infusion | | |
| | 2-4 hours | Belvarafenib PK (plasma) | Matched triplicate |
| | postdose | Cobimetinib PK (plasma) | 12-lead ECG |
| C2D1 | Prior to | Atezolizumab PK (serum) | |
| | infusion | Atezolizumab ADA (serum) | |
| | | Biomarker (Blood for plasma and PBMC) | |
| | Predose | Belvarafenib PK (plasma) | Matched triplicate |
| | | Cobimetinib PK (plasma) | 12-lead ECG |
| C2D15 | Predose | Belvarafenib PK (plasma) | Matched triplicate |
| | | Cobimetinib PK (plasma) | 12-lead ECG |
| | 2-4 hours | Belvarafenib PK (plasma) | Matched triplicate |
| | postdose | Cobimetinib PK (plasma) | 12-lead ECG |
| C3D1 | Prior to | Atezolizumab PK (serum) | |
| | infusion | Atezolizumab ADA (serum) | |
| | Predose | Belvarafenib PK (plasma) | Matched triplicate |
| | | Cobimetinib PK (plasma) | 12-lead ECG |
| Cycles 4, 8, | Prior to | Atezolizumab PK (serum) | |
| and 16, Day 1 | infusion | Atezolizumab ADA (serum) | |
| Cycles 4, 8, | Prior to | Biomarker (Blood for plasma and PBMC) | |
| and 16, Day 1 | infusion | | |

| Treatment discontinuation visit | Belvarafenib PK (plasma) | Matched triplicate |
|---|---|---|
| (≤ 30 days after final dose) | Cobimetinib PK (plasma) | 12-lead ECG |
| | Atezolizumab PK (serum) | |
| | Atezolizumab ADA (serum) | |
| | Biomarker (Blood for | |
| | plasma and PBMC) | |

### PK and Biomarker Evaluation

Belvarafenib concentrations will be analyzed from samples collected at various timepoints as indicated, supra. For the belvarafenib and belvarafenib+cobimetinib arms, the following PK parameters will be derived from the plasma concentrations of belvarafenib versus time from dose using noncompartmental methods, when appropriate for Cycle 1, Day 1 and steady-state: Cₘₐₓ, tₘₐₓ, area under the concentration-time curve (AUC) from nominal time 0 to time t (AUC₀₋ₜ). Furthermore, plasma concentrations of belvarafenib will be reported for all arms as individual values and summarized by treatment arm and cycle, when appropriate and as data allow. Individual and mean belvarafenib concentrations will be plotted by treatment arm and day. Belvarafenib concentration data may be pooled with data from other studies using an established population PK model to derive PK parameters such as clearance, volume of distribution, and AUC, as warranted by the data. Potential correlations of relevant PK parameters with dose, safety, efficacy, or biomarker outcomes may be explored.

Cobimetinib concentrations will be analyzed from samples collected at various timepoints as indicated, supra. For the belvarafenib+cobimetinib arm, the following PK parameters will be derived from the plasma concentrations of cobimetinib using noncompartmental methods, when appropriate for Cycle 1 Day 1 and steady-state: Cₘₐₓ, tₘₐₓ, and AUC₀₋ₜ. For the belvarafenib+cobimetinib arm and the belvarafenib+cobimetinib+atezolizumab arm, plasma concentrations of cobimetinib will be reported as individual values and summarized by treatment arm and cycle, when appropriate and as data allow. Individual and mean cobimetinib concentrations will be plotted by treatment arm and day. Cobimetinib concentration data may be pooled with data from other studies using an established population PK model to derive PK parameters, such as clearance, volume of distribution, and AUC, as warranted by the data. Potential correlations of relevant PK parameters with dose, safety, efficacy, or biomarker outcomes may be explored.

Atezolizumab concentrations will be measured as outlined, supra. Serum concentrations of atezolizumab will be reported as individual values and summarized (mean, standard deviation, coefficient of variation, median, range, geometric mean, and geometric mean coefficient of variation) by treatment arm and cycle, when appropriate and as data allow. Individual and median serum atezolizumab concentrations will be plotted by treatment arm and day. Atezolizumab concentration data may be pooled with data from other studies using an established population PK model to derive PK parameters such as clearance, volume of distribution, and AUC, as warranted by the data. Potential correlations of relevant PK parameters with dose, safety, efficacy, or biomarker outcomes may be explored.

ADA analyses will be conducted on the immunogenicity analysis population for patients enrolled in the belvarafenib+cobimetinib+atezolizumab arm. The immunogenicity analyses will include all patients with at least one ADA assessment for atezolizumab. The numbers and proportions of ADA-positive patients and ADA-negative patients at baseline (baseline prevalence) and after drug administration (postbaseline incidence) will be summarized by treatment group. Summary of baseline prevalence will be based on patients with at least one evaluable baseline ADA assessment; summary of postbaseline incidence will be based on treated patients with at least one evaluable postbaseline ADA assessment. When determining postbaseline incidence, patients are considered to be ADA positive if they are ADA negative or have missing data at baseline but develop an ADA response following study drug exposure (treatment-induced ADA response), or if they are ADA positive at baseline and the titer of one or more postbaseline samples is at least 0.60 titer unit greater than the titer of the baseline sample (treatment-enhanced ADA response). Patients are considered to be ADA negative if they are ADA negative or have missing data at baseline and all postbaseline samples are negative, or if they are ADA positive at baseline but do not have any postbaseline samples with a titer that is at least 0.60 titer unit greater than the titer of the baseline sample (treatment unaffected). The relationship between ADA status and safety, activity, PK, and biomarker endpoints may be analyzed and reported via descriptive statistics.

Biomarker assessments will be performed in an effort to understand the mechanism of action of belvarafenib as a single agent and in combination with cobimetinib and/or atezolizumab, and to identify prognostic and predictive biomarkers to assess disease progression and response to treatment.

Blood samples will be collected at baseline and during the study to evaluate changes in biomarkers, such as biomarkers associated with T cell activation and lymphocyte subpopulations.

To confirm an optimal dose combination of belvarafenib, cobimetinib, and atezolizumab for future studies, the modulation of circulating tumor DNA (ctDNA) will be evaluated to monitor treatment effectiveness. There is increasing evidence that cell-free DNA obtained from blood specimens of patients with cancer contains ctDNA, which is representative of the DNA and mutational status of cells in the tumor (Diehl et al. 2008; Maheswaran et al. 2008). Blood samples will be collected at baseline and during the study to evaluate changes in tumor mutation status in ctDNA as indicators of treatment effect or emergence of resistance to the belvarafenib combinations. Analysis of ctDNA collected before treatment, at various timepoints during treatment, and after a patient progresses on the study may help identify mechanisms of response and acquired resistance to belvarafenib.

Correlations between biomarkers and efficacy endpoints will be explored to identify blood-based biomarkers that might predict which patients are more likely to benefit from the drug combinations of the present disclosure.

Tissue and blood samples will be collected for RNA sequencing analyses and DNA extraction to enable whole genome sequencing (WGS) or whole exome sequencing (WES) to identify variants that are predictive of response to study drug, are associated with acquired resistance to study drug, or can increase the knowledge and understanding of disease biology and to understand the contribution of the immune system to response. WGS and WES provide a comprehensive characterization of the genome and exome, respectively. Together with clinical data collected in this study, WGS and WES may increase the opportunity to develop new therapeutic approaches or new methods for monitoring efficacy and safety or predicting which patients are more likely to respond to a drug or develop adverse events.

To characterize the heterogeneity of tumors among patients and its relationship to clinical response, tumor tissue will be collected at baseline (archival tissues or pretreatment biopsies), during treatment, and at disease progression (fresh biopsy to be done either at first evidence of progression or confirmation of progression, whichever is closest to last date of study treatment administration). Tumor tissue will be evaluated for tumor immunity contextures, such as PD-L1 expression, and other components of tumor immunity. In addition, DNA and/or RNA will be extracted from these tumor samples to enable next-generation sequencing (NGS) of DNA and/or RNA. These molecular characterizations will be analyzed in relation to clinical response to identify patients who will likely benefit more from the treatment. As these biomarkers may also have a prognostic value, their potential association with disease progression will also be explored

Archival tumor tissue or fresh tumor biopsy will also be collected at baseline to enable analysis of tumor tissue biomarkers related to resistance, disease progression, and clinical benefit of subsequent drug treatment during the study. To characterize the heterogeneity of tumors among patients and its relationship to clinical response, tumor tissue will be collected at baseline (archival tissues or pretreatment biopsies), during treatment, and at disease progression (fresh biopsy to be done either at first evidence of progression or confirmation of progression, whichever is closest to last date of study treatment administration). Tumor tissue will be evaluated for tumor immunity contextures, such as PD-L1 expression, and other components of tumor immunity. In addition, DNA and/or RNA will be extracted from these tumor samples to enable next-generation sequencing (NGS) of DNA and/or RNA. These molecular characterizations will be analyzed in relation to clinical response to identify patients who will likely benefit more from the treatment. As these biomarkers may also have a prognostic value, such as to prediction of response and resistance to drug combinations of the present disclosure, and their potential association with disease progression will also be explored. Comparison of biomarkers between tissue acquired before treatment and tissue acquired at the time of progression will further elucidate the potential mechanism of acquired resistance to this combination. Detailed mutation and immune profiles from biopsies taken at disease progression may also provide data for consideration of subsequent therapeutic options.

DNA and RNA sequencing techniques, such as targeted NGS and whole exome, genome, or single nuclei RNA sequencing, may offer a unique opportunity to identify biomarkers of response and/or resistance to belvarafenib when administered alone and in combination with cobimetinib and/or atezolizumab. Sequencing of cancer related genes may result in the identification of de novo and acquired mechanisms of resistance to belvarafenib. NGS technologies can generate a large quantity of sequencing data. Tumor DNA can contain both reported and unreported chromosomal alterations because of the tumorigenesis process. To help control for sequencing calls in previously unreported genomic alterations, a predose blood sample will be taken to determine whether the alteration is somatic. NGS will also be performed on tumor tissue samples to identify immune and stromal signatures and the clonalities of T-cell receptors, in addition to tumor-specific somatic mutations to aid the understanding of disease biology.

### EXAMPLES

The Institutional Animal Care and Use Committee of the Hanmi Research Center approved the animal study protocols of the examples.

### Example 1

NCT03284502 is a Phase 1a/b study investigating belvarafenib in combination with cobimetinib at different dose levels in patients with locally advanced or metastatic solid tumors with RAS or RAF mutation. A total of 67 subjects are enrolled in this study including: 7 subjects receiving 200 mg BID belvarafenib on days 1-28 of a 28-day cycle and 40 mg QD cobimetinib for 21 days of the 28-day cycle; 4 subjects receiving 100 mg BID belvarafenib on days 1-28 of the 28-day cycle and 20 mg QD cobimetinib for 21 days of the 28-day cycle; 27 subjects receiving 200 mg BID belvarafenib on days 1-28 of the 28-day cycle and 20 mg QD cobimetinib for 21 days of the 28-day cycle; 5 subjects receiving 300 mg BID belvarafenib on days 1-28 of the 28-day cycle and 20 mg QD cobimetinib for 21 days of the 28-day cycle; and 24 subjects receiving 300 mg BID belvarafenib on days 1-28 of the 28-day cycle and 20 mg QOD (three time per week) cobimetinib for three weeks of a 28-day cycle.

Dose limiting toxicity (DLT) (including Grade 3 colitis, Grade 3 diarrhea, and Grade 3 nausea) was reported in two out of 3 DLT evaluable subjects treated at belvarafenib 200 mg BID plus cobimetinib 40 mg QD dose level. No DLTs were reported at other dose levels.

61 of 67 subjects (91%) experienced adverse events irrespective of investigators' causality assessment (TEAEs). The TEAEs occurring in ≥10% of subjects were dermatitis acneiform (26 subjects; 38.8%), diarrhea (24 subjects; 35.8%), rash (22 subjects; 32.8%), blood CPK increased (18 subjects; 26.9%), decreased appetite (15 subjects; 22.4%), pyrexia (14 subjects; 20.9%), constipation (13 subjects; 19.4%), anaemia (11 subjects; 16.4%), fatigue (10 subjects; 14.9%), asthenia (8 subjects; 11.9%), and chorioretinopathy (7 subjects; 10.4%). The majority of TEAEs were Grade 1 (5 subjects; 7.5%), or Grade 2 (31 subjects; 46.3%) severity. Grade >3 TEAEs were reported from 25 (37.3%) subjects where events reported in 13 subjects were considered related to both belvarafenib and cobimetinib by the investigators. The most common Grade ≥3 treatment-related adverse events (TRAEs) related to both belvarafenib and cobimetinib were blood CPK increased, fatigue (3 subjects each, 4.5%), and diarrhoea (2 subjects, 3.0%). During the study period, 2 subjects (3.0%) died due to Grade 5 events of cardiac arrest and prolonged anorexia. Grade 5 cardiac arrest occurred in a subject treated with belvarafenib 200 mg BID+cobimetinib 40 mg QD; the event was considered related to both belvarafenib and cobimetinib by the treating physician. Grade 5 event of prolonged anorexia occurred in a subject treated with belvarafenib 300 mg BID+cobimetinib 20 mg QD; the event was considered unrelated to study treatment by the treating physician.

Pharmacokinetic analysis indicated that belvarafenib, in combination with cobimetinib, showed overlapping steady-state exposures (AUC₀₋₂₄) at the 200 mg and 300 mg BID belvarafenib dose. Belvarafenib exposures were within the range of single-agent exposures, indicating no apparent drug-drug interaction with cobimetinib impacting belvarafenib exposure. Cobimetinib steady-state exposures were consistent with dose normalized steady-state single agent cobimetinib exposures, indicating no apparent drug-drug interaction with belvarafenib impacting cobimetinib exposure.

### Example 2

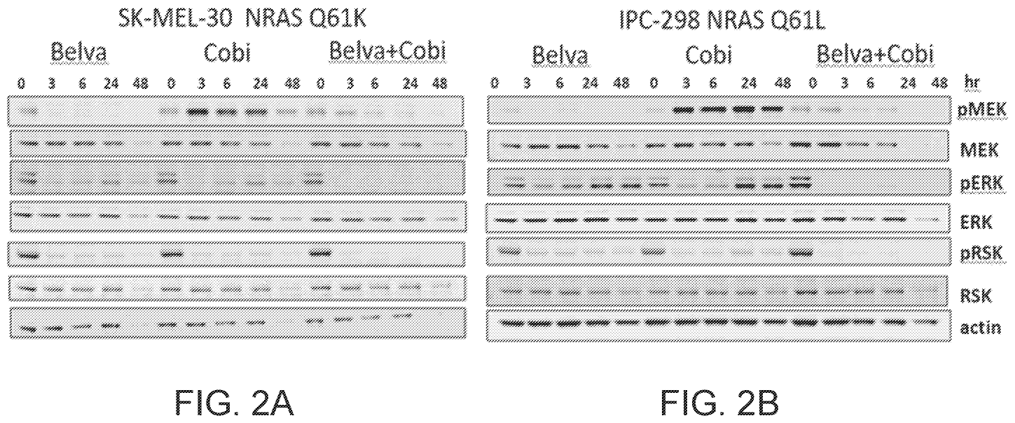

In example 2, in vitro signaling of belvarafenib, cobimetinib, and their combination in NRAS mutant melanoma cells was evaluated. SK-MEL-30 (NRAS^{Q61K}) and IPC-298 (NRAS^{Q61L}) melanoma cells were treated with 1 uM belvarafenib (Belva), 250 nM cobimetinib (Cobi), or the combination of both compounds (Belva+Cobi) and were evaluated at 0, 3, 6, 24, and 48 hours. The cells were analyzed for MAPK signaling markers (phosphorylated MEK, ERK, and RSK) by western blot. The results for SK-MEL-30 (NRAS^{Q61K}) are shown in FIG. 2A and the results for IPC-298 (NRAS^{Q61L}) are shown in FIG. 2B. The results show that the combination of belvarafenib and cobimetinib inhibits MAPK pathway signaling in NRAS mutant cell lines.

### Example 3

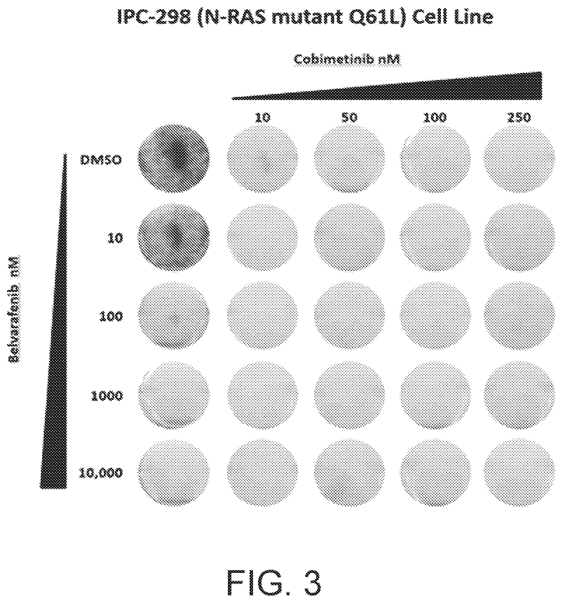

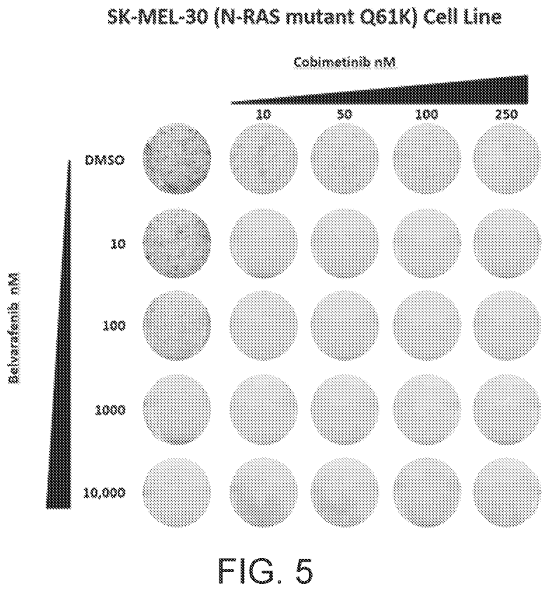

In Example 3, an in vitro colony formation growth study of belvarafenib, cobimetinib, or their combination in melanoma cells was evaluated. The following combinations were evaluated: 10 nM cobi+vehicle; 50 nM cobi+vehicle; 100 nM cobi+vehicle; 250 nM cobi+vehicle; 10 nM belv+vehicle; 100 nM belv+vehicle; 1000 nM belv+vehicle; 10,000 nM belv+vehicle; 10 nM cobi+10 nM belv; 10 nM cobi+100 nM belv; 10 nM cobi+1000 nM belv; 10 nM cobi+10,000 nM belv; 50 nM cobi+10 nM belv; 50 nM cobi+100 nM belv; 50 nM cobi+1000 nM belv; 50 nM cobi+10,000 nM belv; 100 nM cobi+10 nM belv; 100 nM cobi+100 nM belv; 100 nM cobi+1000 nM belv; 100 nM cobi+10,000 nM belv; 250 nM cobi+10 nM belv; 250 nM cobi+100 nM belv; 250 nM cobi+1000 nM belv; and 250 nM cobi+10,000 nM belv. The following cell lines were evaluated: IPC-298 (NRAS^{Q61L}) melanoma cell line; Mel-Juso (NRAS^{Q61L}) melanoma cell line treated with belvarafenib, cobimetinib, or their combination; and SK-MEL-30 (NRAS^{Q61K}) melanoma cell line. The cells were treated with the indicated concentrations of belvarafenib and/or cobimetinib for 8 days, followed by staining with crystal violet. The results are depicted in FIGS. 3-5. The results show that the combination of belvarafenib and cobimetinib inhibits cell colony formation in NRAS mutant cell lines.

### Example 4

The in vitro signaling of the pan RAF inhibitor belvarafenib was evaluated versus the BRAF inhibitors vemurafenib and dabrafenib in KRAS mutant CRC cell lines HCT116 (KRAS^{G13D}) and Lovo (KRAS^{G13D}) and in NSCLC cell line Calu-6 (KRAS^{Q61K}) To determine the IC₅₀ in nM, the cell lines were exposed to belvarafenib, vemurafenib, and dabrafenib over a concentration range and evaluated at 2 hours after exposure.

The results are presented in Table 5 below. As shown in Table 5, belvarafenib, but not vemurafenib and dabrafenib, showed inhibitory effects on the phosphorylation of MEK and ERK in HCT116, Lovo, and Calu-6 cell lines. In vitro cellular IC₅₀ values of belvarafenib for MEK and ERK phosphorylation were 2,698 and 253 nM, respectively, in HCT116; >10 μM (37% inhibition at 10 μM) and 267 nM in Lovo; and 367 and 590 nM in Calu-6 cell lines, respectively. The corresponding IC₅₀ values for vemurafenib and dabrafenib were >10 μM in HCT116, Lovo, and Calu-6 cell lines.

TABLE 5
Inhibition of MEK and ERK Phosphorylation
by Belvarafenib, Vemurafenib and Dabrafenib
in KRAS Mutant CRC and NSCLC Cell Lines

| | | | | IC₅₀ (nM), 2 hr | |
|---|---|---|---|---|---|
| | Cell | KRAS | | after treatment | |
| Cancer | line | mutation | Compound | pMEK | pERK |
| CRC | HCT116 | G13D | Belvarafenib | 2,698 | 253 |
| | | | Vemurafenib | >10,000 | >10,000 |
| | | | Dabrafenib | >10,000 | >10,000 |
| | Lovo | G13D | Belvarafenib | >10,000^{a} | 267 |
| | | | Vemurafenib | >10,000 | >10,000 |
| | | | Dabrafenib | >10,000 | >10,000 |
| NSCLC | Calu-6 | Q61K | Belvarafenib | 367 | 590 |
| | | | Vemurafenib | >10,000 | >10,000 |
| | | | Dabrafenib | >10,000 | >10,000 |

^{a}Belvarafenib inhibited phosphorylation of MEK at 37% at 10 μM.

### Example 5

The in vitro cell growth inhibition of BRAF or KRAS Mutant CRC and KRAS mutant NSCLC cell lines by belvarafenib was evaluated versus the BRAF inhibitors vemurafenib and dabrafenib in: BRAF mutant CRC cell lines HT-29 and Colo-205 (both BRAF^{V600E}); KRAS mutant CRC cell lines LS174T (KRAS^{G12D}), LS513 (KRAS^{G12D}) HCT116 (KRAS^{G13D}) and Lovo (KRAS^{G13D}); and KRAS mutant NSCLC cell lines Calu-6 (KRAS^{Q61K}) and Calu-1 (KRAS^{G12C}).

The results are presented in Tables 6 and 7 below. While belvarafenib and vemurafenib showed comparable activity on cell growth inhibition in BRAF mutant CRC cell lines, HT-29 and Colo-205 (GI₅₀ range=47-118 nM), dabrafenib showed the most potent cell growth inhibitory effect in those cells with GI₅₀<0.1 nM. Belvarafenib inhibited cell growth in all KRAS mutant CRC cell lines tested in vitro, including: LS174T, LS513, HCT116 and Lovo with GI₅₀ values of 258, 62, 177 and 51 nM, respectively (Table 6). Activity of belvarafenib on cell growth inhibition of KRAS mutant NSCLC cell lines was also observed in Calu-6 and Calu-1 (GI₅₀, 179 and 749 nM, respectively) (Table 7). Dabrafenib also showed in vitro cell growth inhibition in Lovo (KRAS mutant, CRC) cell line (GI₅₀=214 nM), and Calu-6 and Calu-1 (KRAS mutant, NSCLC) cell lines (GI₅₀, 618 and 904 nM, respectively). The activities of dabrafenib, however, were about 3 to 4-fold weaker than belvarafenib, except in Calu-1 cells. Vemurafenib showed no activity on the inhibition of growth in KRAS mutant cells.

TABLE 6
In vitro Cell Growth Inhibition of BRAF or KRAS
Mutant Colorectal Cancer (CRC) Cell Lines by
Belvarafenib versus Vemurafenib and Dabrafenib

| | | GI₅₀ (nM) | | |
|---|---|---|---|---|
| Cell line | Mutation | Belvarafenib | Vemurafenib | Dabrafenib |
| HT-29 | BRAF^{V600E} | 47 | 110 | <0.1 |
| Colo-205 | BRAF^{V600E} | 118 | 65 | <0.1 |
| LS174T | KRAS^{G12D} | 258 | >10,000 | >10,000 |
| LS513 | KRAS^{G12D} | 62 | >10,000 | 3,059 |
| HCT116 | KRAS^{G13D} | 177 ± 103 | >10,000 | >10,000 |
| Lovo | KRAS^{G13D} | 51 ± 19 | 4,025 ± 117 | 214 ± 61 |

TABLE 7
In vitro Cell Growth Inhibition of KRAS Mutant
Non-Small Cell Lung Cancer (NSCLC) Cell Lines
by Belvarafenib versus Vemurafenib and Dabrafenib

| | KRAS | GI₅₀ (nM) | | |
|---|---|---|---|---|
| Cell line | Mutation | Belvarafenib | Vemurafenib | Dabrafenib |
| Calu-6 | Q61K | 179 ± 103 | >10,000 | 618 ± 252 |
| Calu-1 | G12C | 749 | 7,476 | 904 |

### Example 6

The in vitro cell growth inhibitory activity of belvarafenib versus other BRAF inhibitors, vemurafenib and dabrafenib, on BRAF or KRAS mutant cell lines was further investigated in BRAF mutant thyroid cell lines: SNU790, FRO, B-CPAP, NPA, 8505C, ARO (all BRAF^{V600E}) and SNU80 (BRAFG^{468R}); and KRAS mutant thyroid cancer cell line, CAL-62 (KRAS^{G12R}). The results are reported in Table 8.

Belvarafenib and dabrafenib showed activity on cell growth inhibition in all 7 BRAF mutant thyroid cancer cell lines (GI₅₀, <1 μM). Vemurafenib showed cell growth inhibitory effect in SNU790, B-CPAP and NPA, BRAF mutant thyroid cancer cell lines, with GI₅₀ values <1 μM. Additionally, only belvarafenib, but not vemurafenib or dabrafenib, showed activity on cell growth inhibition in CAL-62 (KRAS^{G12R}) thyroid cancer cells, with GI₅₀ value of 479 nM.

TABLE 8
In vitro Cell Growth Inhibition of BRAF or
KRAS Mutant Thyroid Cancer Cell Lines by Belvarafenib
versus Vemurafenib and Dabrafenib

| | | GI₅₀ (nM) | | |
|---|---|---|---|---|
| Cell Line | Mutation | Belvarafenib | Vemurafenib | Dabrafenib |
| SNU7890 | BRAF^{V600E} | 27 | 190 | 0.1 |
| FRO | BRAF^{V600E} | 30 | 1,763 | 23 |
| B-CPAP | BRAF^{V600E} | 43 | 31 | <0.1 |
| NPA | BRAF^{V600E} | 45 | 44 | <0.1 |
| 8505C | BRAF^{V600E} | 345 | 3,444 | 184 |
| ARO | BRAF^{V600E} | 719 | 5,260 | 548 |
| SNU80 | BRAF^{G468R} | 163 | 2,848 | 51 |
| CAL-62 | KRAS^{G12R} | 479 | >10,000 | >10,000 |

### Example 7

The in vivo antitumor activity of the combination of belvarafenib and cobimetinib as a combination therapy in a mice model xenografted with SK-MEL-30 human melanoma cell line harboring NRAS^{Q61K} mutation was investigated. Five animals per group were treated with the vehicle (control), belvarafenib alone at a dose of 10 or 30 mg/kg once daily, cobimetinib alone at a dose of 10 mg/kg once daily, and their combination therapies once daily via oral gavage up to day 14. Tumor volume in mm³ and body weight change were measured at days 1, 4, 9 and 15.

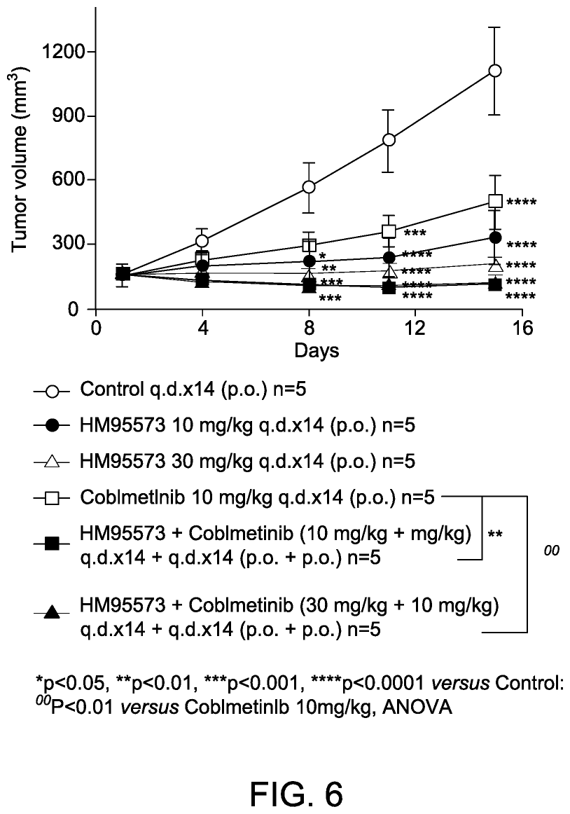

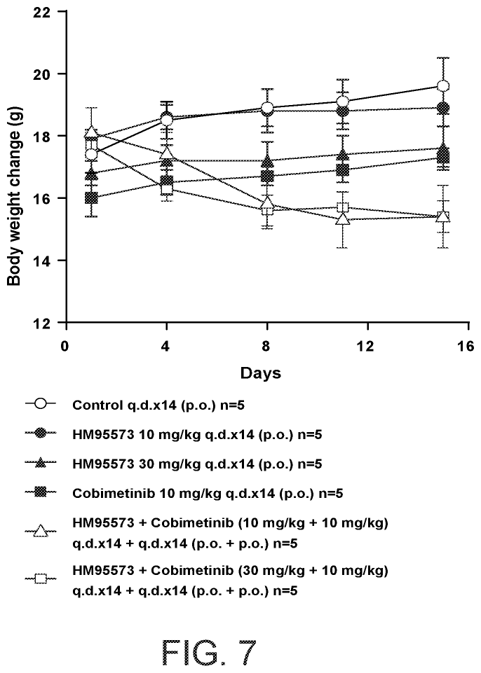

As shown in FIGS. 6 and 7, and Table 9, oral administration of belvarafenib resulted in dose-dependent antitumor activity with 70.3% (on day 15), and 80.0% (on day 15) of the maximum inhibition rate at 10 and 30 mg/kg q.d., respectively. Maximum inhibition rate (max inhib (%)) was determined by (1−(mean relative tumor weight in treated group/mean relative tumor weight control group))*100. Cobimetinib alone given at 10 mg/kg q.d. showed maximum inhibition rate of 54.3% (on day 15). Both treatment with belvarafenib and cobimetinib were well tolerated without body weight loss; for belvarafenib-treatment groups, clinical sign of hair growth on the back was observed. Combination therapies of belvarafenib 10 or 30 mg/kg q.d. and cobimetinib 10 mg/kg q.d. showed additive antitumor activity with a maximum inhibition rate of 89.8% (on day 15) or 89.1% (on day 15), respectively. Mortality or significant body weight loss (>10%) were also observed. Combination therapies of belvarafenib at 10 or 30 mg/kg q.d. and cobimetinib at 10 mg/kg q.d. showed additive antitumor activity with a maximum inhibition rate of 89.8% (on day 15) or 89.1% (on day 15), respectively. Mortality or significant body weight loss (>10%) were also observed.

TABLE 9
In vivo Antitumor Activities of Belvarafenib, or Cobimetinib Alone
or Their Combination Administered Orally for 14 days in Mice
Xenografted with SK-MEL-30 Melanoma Cancer Cell Line

| | | | | Max | Day | Max |
|---|---|---|---|---|---|---|
| | | | | inhib. | for | weight |
| | Dose | | | Rate | max | loss |
| Compound | (mg/kg) | Regimen | Duration | (%) | inhib. | (%) |
| Belvarafenib | 10 | QD | 14 days | 70.3 | Day 15 | — |
| | 30 | QD | 14 days | 80.0 | Day 15 | — |
| Cobimetinib | 10 | QD | 14 days | 54.3 | Day 15 | — |
| Belvarafenib + | 10 + 10 | QD, QD | 14 days | 89.8 | Day 15 | 15.7 |
| Cobimetinib | 30 + 10 | QD, QD | 14 days | 89.1 | Day 15 | 13.1 |

### Example 8

The in vivo antitumor activity of belvarafenib in mice harboring NRAS-mutant patient-derived xenograft (PDX) was investigated. Ten animals per group were treated with the vehicle (control) or belvarafenib at a dose of 20 mg/kg QD. Tumor volume and body weight change were measured every 3-4 days. As shown in Tables 10 to 12C, oral administration of belvarafenib resulted in strong antitumor activity, and belvarafenib treatment was well tolerated without significant (>10%) body weight loss. The data in Tables 11A to 12C was converted from graphical form with an estimated error of +/−10% (e.g., +/−20 mm3 for Tables 11A to 11C).

The overall results for Example 8 are presented in Table 10 below.

TABLE 10

| | NRAS | | Maximum |
|---|---|---|---|
| PDX model# | Mutation | Duration | TGI |
| Model# 1 | Q61K | 28 | 85% |
| Model# 2 | Q61R | 20 | 107% |
| Model# 3 | G12C | 21 | 90% |
| Model# 4 | Q61R | 20 | 127% |
| Model# 5 | Q61R | 20 | 130% |
| Model# 6 | Q61K | 20 | 110% |
| Model# 7 | Q61R | 21 | 124% |
| Model# 8 | Q61R | 20 | 115% |
| Model# 9 | Q61K | 20 | 125% |

Tables 11A-11C: Tumor volume results of efficacy studies of NTRAS PDX melanoma models. For each PDX model, the belvarafenib dose was 20 mg/kg QD.

TABLE 11A

| | PDX Model# 1 | | PDX Model# 2 | | PDX Model# 3 | |
|---|---|---|---|---|---|---|
| Day | belvarafenib | vehicle | belvarafenib | vehicle | belvarafenib | vehicle |
| 0 | 240 mm³ | 240 mm³ | 210 mm³ | 210 mm³ | 200 mm³ | 200 mm³ |
| 2 | 250 mm³ | 300 mm³ | 220 mm³ | 270 mm³ | 240 mm³ | 240 mm³ |
| 6 | 320 mm³ | 460 mm³ | 230 mm³ | 400 mm³ | 300 mm³ | 360 mm³ |
| 9 | — | — | 200 mm³ | 490 mm³ | 300 mm³ | 480 mm³ |
| 10 | 350 mm³ | 580 mm³ | — | — | — | — |
| 13 | — | — | 160 mm³ | 580 mm³ | — | — |
| 14 | 320 mm³ | 670 mm³ | — | — | 240 mm³ | 640 mm³ |
| 16 | — | — | 120 mm³ | 690 mm³ | — | — |
| 17 | 300 mm³ | 810 mm³ | — | — | 220 mm³ | 760 mm³ |
| 20 | 290 mm³ | 1050 mm³ | 100 mm³ | 1000 mm³ | — | — |
| 21 | — | — | — | — | 200 mm³ | 920 mm³ |
| 23 | 280 mm³ | 1300 mm³ | — | — | — | — |
| 28 | 260 mm³ | 1600 mm³ | — | — | — | — |

TABLE 11B

| | PDX Model# 4 | | PDX Model# 5 | | PDX Model# 6 | |
|---|---|---|---|---|---|---|
| Day | belvarafenib | vehicle | belvarafenib | vehicle | belvarafenib | vehicle |
| 0 | 220 mm³ | 220 mm³ | 200 mm³ | 200 mm³ | 200 mm³ | 200 mm³ |
| 2 | 180 mm³ | 280 mm³ | — | — | 210 mm³ | 240 mm³ |
| 3 | — | — | 180 mm³ | 250 mm³ | — | — |
| 6 | 160 mm³ | 400 mm³ | 160 mm³ | 320 mm³ | 200 mm³ | 310 mm³ |
| 9 | 140 mm³ | 480 mm³ | — | — | 195 mm³ | 370 mm³ |
| 10 | — | — | 130 mm³ | 400 mm³ | — | — |
| 13 | 120 mm³ | 560 mm³ | 110 mm³ | 460 mm³ | 190 mm³ | 480 mm³ |
| 16 | 110 mm³ | 640 mm³ | — | — | 185 mm³ | 600 mm³ |
| 17 | — | — | 90 mm³ | 600 mm³ | — | — |
| 20 | 90 mm³ | 800 mm³ | 80 mm³ | 770 mm³ | 185 mm³ | 780 mm³ |

TABLE 11C

| | PDX Model# 7 | | PDX Model# 8 | | PDX Model# 9 | |
|---|---|---|---|---|---|---|
| Day | belvarafenib | vehicle | belvarafenib | vehicle | belvarafenib | vehicle |
| 0 | 200 mm³ | 200 mm³ | 200 mm³ | 200 mm³ | 200 mm³ | 200 mm³ |
| 2 | 190 mm³ | 270 mm³ | — | — | 195 mm³ | 240 mm³ |
| 3 | — | — | 180 mm³ | 300 mm³ | — | — |
| 6 | 140 mm³ | 410 mm³ | — | — | 180 mm³ | 320 mm³ |
| 7 | — | — | 160 mm³ | 450 mm³ | — | — |
| 9 | 100 mm³ | 550 mm³ | — | — | 160 mm³ | 400 mm³ |
| 10 | — | — | 150 mm³ | 580 mm³ | — | — |
| 13 | — | — | — | — | 120 mm³ | 480 mm³ |
| 14 | 70 mm³ | 850 mm³ | 140 mm³ | 750 mm³ | — | — |
| 16 | — | — | — | — | 100 mm³ | 560 mm³ |
| 17 | 50 mm³ | 1050 mm³ | 130 mm³ | 890 mm³ | — | — |
| 20 | — | — | 120 mm³ | 1000 mm³ | 80 mm³ | 680 mm³ |
| 21 | 50 mm³ | 1300 mm³ | — | — | — | — |

Tables 12A-12C: Body weight change (%) results of efficacy studies of NRAS PDX melanoma models. For each PDX model, the belvarafenib dose was 20 mg/kg QD.

TABLE 12A

| | PDX Model# 1 | | PDX Model# 2 | | PDX Model# 3 | |
|---|---|---|---|---|---|---|
| Day | belvarafenib | vehicle | belvarafenib | vehicle | belvarafenib | vehicle |
| 0 | 0% | 0% | 0% | 0% | 0% | 0% |
| 2 | −0.6% | −3.1% | −1.5% | −1.5% | 1.6% | 1.6% |
| 6 | −1.6% | −7.5% | −3.8% | −5.3% | 4.3% | 3.6% |
| 9 | — | — | −4% | −7.4% | 6.1% | 2.9% |
| 10 | −2.2% | −8.9% | — | — | — | — |
| 13 | — | — | −2.5% | −8.8% | — | — |
| 14 | −1.9% | −7.2% | — | — | 8.2% | −0.4% |
| 16 | — | — | −.6% | −8.2% | — | — |
| 17 | −1.2% | −5% | — | — | 9.1% | −1.2% |
| 20 | −.3% | −2.7% | 2.6% | −5.6% | — | — |
| 21 | — | — | — | — | 10% | 0.2% |
| 23 | 1.3% | −.6% | — | — | — | — |
| 28 | 4.1% | 2.7% | — | — | — | — |

TABLE 12B

| | PDX Model# 4 | | PDX Model# 5 | | PDX Model# 6 | |
|---|---|---|---|---|---|---|
| Day | belvarafenib | vehicle | belvarafenib | vehicle | belvarafenib | vehicle |
| 0 | 0% | 0% | 0% | 0% | 0% | 0% |
| 2 | −2.8% | −2.8% | — | — | −7.2% | −1.1% |
| 3 | — | — | −2.3% | −0.9% | — | — |
| 6 | −4.6% | −7.4% | 0.3% | 0% | −12.3% | −10.6% |
| 9 | −2.2% | −8.9% | — | — | −122.2% | −11.5% |
| 10 | — | — | −1.6% | −4% | — | — |
| 13 | 3.3% | −8.1% | 3.6% | −6.9% | −9.6% | −11.1% |
| 16 | 4.8% | −5.9% | — | — | −9.6% | −10.2% |
| 17 | — | — | 7.2% | −5.6% | — | — |
| 20 | 1.9% | −1.9% | 3.4% | −8.6% | −9.6% | −10.2% |

TABLE 12C

| | PDX Model# 7 | | PDX Model# 8 | | PDX Model# 9 | |
|---|---|---|---|---|---|---|
| Day | belvarafenib | vehicle | belvarafenib | vehicle | belvarafenib | vehicle |
| 0 | 0% | 0% | 0% | 0% | 0% | 0% |
| 2 | 1.4% | 0.3% | — | — | −1.2% | 0.2% |
| 3 | — | — | −0.8% | 0.1% | — | — |
| 6 | 4.3% | −0.1% | — | — | 3.7% | 0.6% |
| 7 | — | — | −1% | 0.4% | — | — |
| 9 | 5.7% | −1.7% | — | — | 8.4% | −0.2% |
| 10 | — | — | −0.4% | 0.6% | — | — |
| 13 | — | — | — | — | 10.5% | −0.2% |
| 14 | 7.1% | −5.4% | 0.6% | 1% | — | — |
| 16 | — | — | — | — | 13.5% | −0.6% |
| 17 | 7.6% | −6.9% | 1.2% | 1.15% | — | — |
| 20 | — | — | 1.35% | 1.3% | 14.9% | 2.1% |
| 21 | 8.3% | −7.3% | — | — | — | — |

### Example 9

In vivo Combination Study of Belvarafenib with the MEK inhibitors MEK162 (binimetinib) in a SK-MEL-30 Xenograft Model.

MEK inhibitors as monotherapy appear to provide minimal benefit in patients previously treated with a BRAF inhibitor. Combined BRAF and MEK inhibitors in NRAS mutant tumors may prevent or overcome resistance to monotherapy and potentially improve the safety profile of single-agent therapy. In this respect, in vivo antitumor activity of belvarafenib as a combination therapy with binimetinib|, a selective ATP-uncompetitive inhibitor of MEK1/2 was investigated in mice model xenografted with SK-MEL-30 melanoma cell line harboring NRAS^{Q61K} mutation. Five animals per group were treated with vehicle (control), belvarafenib alone at a dose of 10, or 30 mg/kg once daily, binimetinib alone at a dose of 10 or 30 mg/kg once daily, or their combination (belvarafenib 10 mg/kg+binimetinib 10 mg/kg or belvarafenib 30 mg/kg+binimetinib 30 mg/kg) once daily via oral gavage for 21 days.

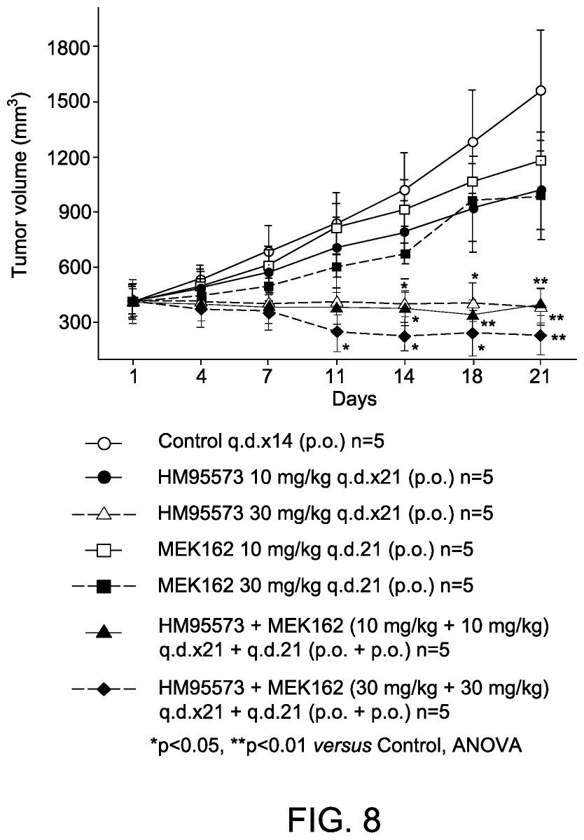

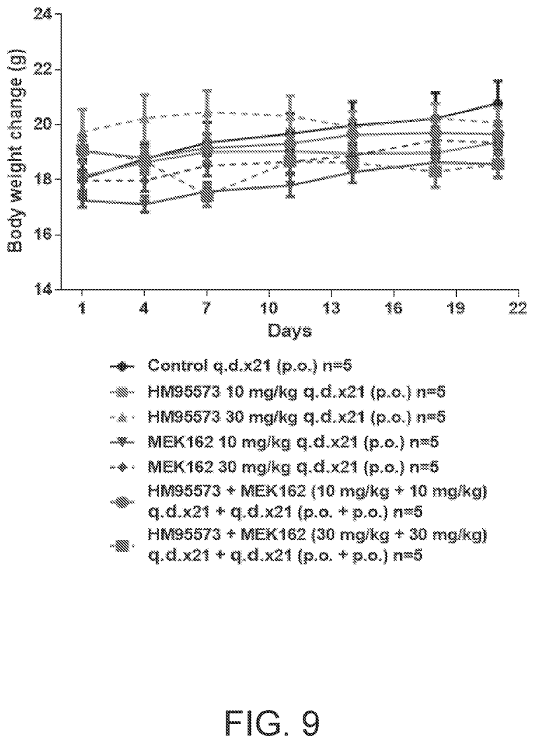

As shown in FIGS. 8 and 9, and Table 13, oral administration of belvarafenib resulted in dose-dependent antitumor activity with 36.7% (on day 21), and 74.6% (on day 21) of the maximum inhibition rate at 10 and 30 mg/kg q.d., respectively. Also, binimetinib given at 10 and 30 mg/kg q.d. showed antitumor activity on this tumor model with maximum inhibition rate of 13.6% (on day 21) and 27.1% (on day 21), respectively. Both treatment with belvarafenib and binimetinib were well tolerated without marked body weight loss; for belvarafenib-treatment group, clinical signs of hair growth on the back, abdomen and facial area were observed at 10 mg/kg dose or above.

Combination therapy of belvarafenib 10 mg/kg q.d. and binimetinib 10 mg/kg q.d. showed enhanced antitumor activity with maximum inhibition rate of 74.2% on day 21 and toxicity of edema or erythema on face, which was not observed in either treatment alone. Belvarafenib at 30 mg/kg q.d. co-administered with binimetinib 30 mg/kg q.d. induced tumor shrinkage (77.7% on day 21) but was poorly tolerated; 2/5 animals were found dead on day 10 and other survived animals showed clinical signs such as edema or erythema on face which was not observed in either treatment alone during the study period.

TABLE 13
In vivo Antitumor Activities of Belvarafenib, or Binimetinib
Either Alone or Combination of Belvarafenib and Binimetinib
Administered Orally for 21 Days in Mice Xenografted with
SK-MEL-30 Melanoma Cell Line

| | | | | Max | Day | Max |
|---|---|---|---|---|---|---|
| | | | | inhib. | for | weight |
| | Dose | | | Rate | max | loss |
| Compound | (mg/kg) | Regimen | Duration | (%) | inhib | (%) |
| Belvarafenib | 10 | QD | 21 days | 36.7 | Day 21 | — |
| | 30 | QD | 21 days | 74.6 | Day 21 | — |
| Binimetinib | 10 | QD | 21 days | 13.6 | Day 21 | 0.8 |
| | 30 | QD | 21 days | 27.1 | Day 21 | — |
| Belvarafenib + | 10 + 10 | QD, QD | 21 days | 74.2 | Day 21 | 1.3 |
| Binimetinib | 30 + 30 | QD, QD | 21 days | 77.7 | Day 21 | 8.7 |

### Example 10

In vivo Combination Study of Belvarafenib with AZD6244 in Calu-6 Xenograft Model.

In vivo antitumor activity of belvarafenib as a combination therapy with selumetinib (AZD6244), a selective ATP-uncompetitive inhibitor of MEK1/2, was investigated in mice model xenografted with Calu-6 NSCLC cell line harboring KRAS^{Q61K} mutation. Five animals per group were treated with vehicle (control), belvarafenib alone at a dose of 3, 10, or 30 mg/kg once daily, selumetinib alone at a dose of 5 mg/kg twice daily, or their combination therapy (belvarafenib 3 or 10 mg/kg once daily+selumetinib 5 mg/kg twice daily) via oral gavage for 17 days.

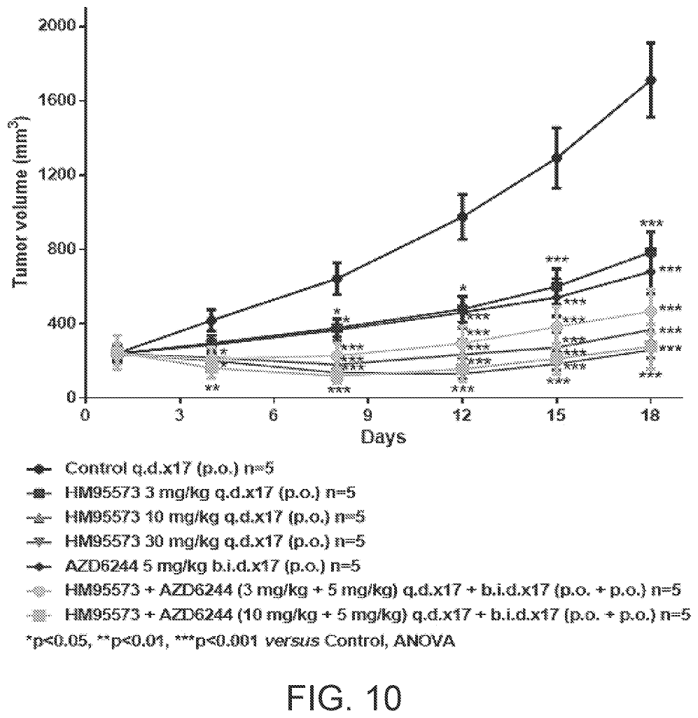

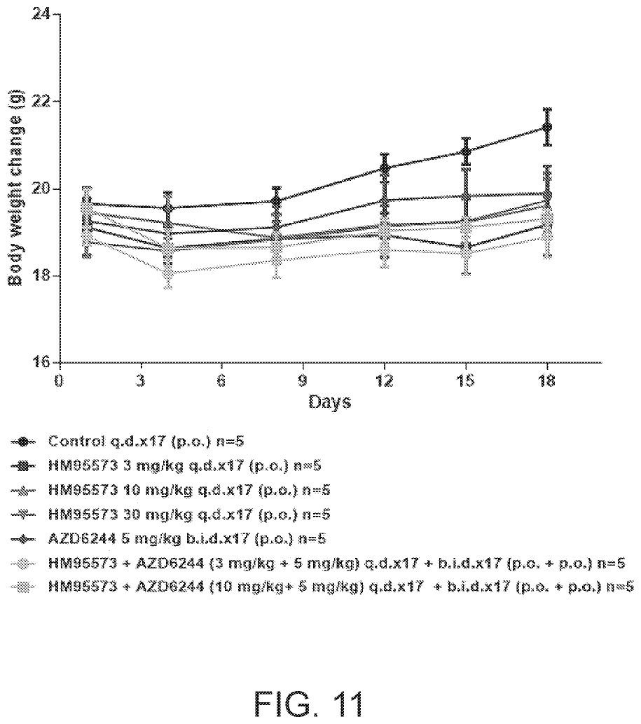

As shown in FIGS. 10 and 11 and Table 14, oral administration of belvarafenib resulted in dose-dependent antitumor activity with 53.5% (on day 18), 79.3% (on day 15) and 86.3% (on day 12) of the maximum inhibition rate at 3, 10, and 30 mg/kg q.d., respectively. Also, selumetinib given at 5 mg/kg b.i.d. showed significant antitumor activity on this tumor model with maximum inhibition rate of 59.0% on day 18. Either treatment with belvarafenib or selumetinib was well tolerated with or without minimal body weight loss; for belvarafenib-treatment group, clinical signs of hair growth on the back, abdomen and facial area were observed at 10 mg/kg dose or above. Combination therapy of belvarafenib and selumetinib resulted in enhanced antitumor activity without further toxicity observed in either treatment alone; maximum inhibition rate from belvarafenib 3 or 10 mg/kg q.d. and selumetinib 5 mg/kg b.i.d. was 71.8% (on day 18) and 85.5% (on day 12), respectively.

TABLE 14

| | | | | Max | Day | Max |
|---|---|---|---|---|---|---|
| | | | | inhib. | for | weight |
| | Dose | | | Rate | max | loss |
| Compound | (mg/kg) | Regimen | Duration | (%) | inhib | (%) |
| Belvarafenib | 3 | QD | 17 days | 53.5 | Day 18 | 2.5 |
| | 10 | QD | 17 days | 79.3 | Day 15 | 3.0 |
| | 30 | QD | 17 days | 86.3 | Day 12 | 1.1 |
| Selumetinib | 5 | BID | 17 days | 59.0 | Day 18 | 1.5 |
| Belvarafenib + | 3 + 5 | QD, BID | 17 days | 71.8 | Day 18 | 4.5 |
| Selumetinib | 10 + 5 | QD, BID | 17 days | 85.5 | Day 12 | 4.7 |

### Example 11

The combination therapies of (i) belvarafenib and cobimetinib and (ii) belvarafenib, cobimetinib and atezolizumab will be evaluated in a clinical study according to the protocol depicted in FIG. 1 and described, supra.

This written description uses examples to disclose the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of treating a subject having NRAS-mutant melanoma, the method comprising: (i) administering to said subject a therapy consisting essentially of (ii) a therapeutically effective amount of belvarafenib or a pharmaceutically acceptable salt thereof and (iii) a therapeutically effective amount of cobimetinib or a pharmaceutically acceptable salt thereof.

2. The method of claim 1 wherein the melanoma carries a NRAS^{Q61K} mutation, a NRAS^{Q61R} mutation, a NRAS^{G12C} mutation, NRAS^{Q61L} mutation, a NRAS^{G13D} mutation, or any combination thereof.

3. The method of claim 1 wherein the melanoma carries a NRAS^{G12D} mutation, a NRAS^{G12C} mutation, or a combination thereof.

4. The method of any one of claims 1 to 3, wherein the melanoma is metastatic or unresectable.

5. The method of any one of claims 1 to 4, wherein the subject is administered from about 200 mg to about 1300 mg, from about 400 mg to about 1200 mg, from about 600 mg to about 1200 mg, or from about 800 mg to about 1000 mg, of belvarafenib or a pharmaceutically acceptable salt thereof per day.

6. The method of any one of claims 1 to 5, wherein the subject is administered about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, or about 1300 mg of belvarafenib or a pharmaceutically acceptable salt thereof per day.

7. The method of claim 6, wherein the subject is administered about 250 mg, about 300 mg, about 350 mg, about 400 mg, about 450 mg, or about 500 mg of belvarafenib or a pharmaceutically acceptable salt thereof twice per day.

8. The method of claim 7, wherein the subject is administered about 300 mg or about 400 mg of belvarafenib or a pharmaceutically acceptable salt thereof twice per day.

9. The method of any one of claims 1 to 8, wherein belvarafenib or a pharmaceutically acceptable salt thereof is administered daily for 28 consecutive days of a 28-day treatment cycle.

10. The method of any one of claims 1 to 9, wherein belvarafenib or a pharmaceutically acceptable salt thereof is administered with food.

11. The method of any one of claims 1 to 10, wherein the subject is administered from about 20 mg to about 100 mg of cobimetinib or a pharmaceutically acceptable salt thereof per day.

12. The method of claim 11, wherein the subject is administered about 20 mg, about 40 mg or about 60 mg of cobimetinib or a pharmaceutically acceptable salt thereof per day.

13. The method of any one of claims 1 to 12, wherein cobimetinib or a pharmaceutically acceptable salt thereof is administered daily for 21 consecutive days of a 28-day treatment cycle.

14. The method of claim 13, wherein cobimetinib or a pharmaceutically acceptable salt thereof is administered on days 1 to 21 of a 28-day treatment cycle.

15. The method of claim 13, wherein cobimetinib or a pharmaceutically acceptable salt thereof is administered on days 3 to 23 of a 28-day treatment cycle.

16. The method of any one of claims 1 to 15, wherein the subject was previously administered a course of treatment with an anti-PD-1 drug or anti-PD-L1 drug.

17. The method of any one of claims 1 to 16, wherein the subject experienced disease progression after treatment with immunotherapy, BRAF V600E therapy, or a combination of immunotherapy and BRAF V600E therapy.

18. The method of any one of claims 1 to 17, wherein said method for treating cancer is characterized by the absence of the development of squamous cell carcinoma in the human subject.

19. A method of treating a subject having NRAS-mutant melanoma, the method comprising: (i) administering to said subject a therapy consisting essentially of (ii) a therapeutically effective amount of belvarafenib or a pharmaceutically acceptable salt thereof, (iii) a therapeutically effective amount of cobimetinib or a pharmaceutically acceptable salt thereof, and (iv) a therapeutically effective amount of atezolizumab.

20. The method of claim 19 wherein the melanoma carries a NRAS^{Q61K} mutation, a NRAS^{Q61R} mutation, a NRAS^{G12C} mutation, NRAS^{Q61L} mutation, a NRAS^{G13D} mutation, or any combination thereof.

21. The method of claim 19, wherein the melanoma carries a NRAS^{G12D} mutation, a NRAS^{G12C} mutation, or a combination thereof.

22. The method of any one of claims 19 to 21, wherein the melanoma is metastatic or unresectable.

23. The method of any one of claims 19 to 22, wherein the subject is administered from about 200 mg to about 1300 mg, from about 400 mg to about 1200 mg, from about 600 mg to about 1200 mg, or from about 800 mg to about 1000 mg, of belvarafenib or a pharmaceutically acceptable salt thereof per day.

24. The method of any one of claims 19 to 23, wherein the subject is administered about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, or about 1300 mg of belvarafenib or a pharmaceutically acceptable salt thereof per day.

25. The method of claim 24, wherein the subject is administered about 250 mg, about 300 mg, about 350 mg, about 400 mg, about 450 mg, or about 500 mg of belvarafenib or a pharmaceutically acceptable salt thereof twice per day.

26. The method of claim 25, wherein the subject is administered about 300 mg or about 400 mg of belvarafenib or a pharmaceutically acceptable salt thereof twice per day.

27. The method of any one of claims 19 to 26, wherein belvarafenib or a pharmaceutically acceptable salt thereof is administered daily for 28 consecutive days of a 28-day treatment cycle.

28. The method of any one of claims 19 to 27, wherein belvarafenib or a pharmaceutically acceptable salt thereof is administered with food.

29. The method of any one of claims 19 to 28, wherein the subject is administered from about 20 mg to about 100 mg of cobimetinib or a pharmaceutically acceptable salt thereof per day.

30. The method of claim 29, wherein the subject is administered about 20 mg, about 40 mg or about 60 mg of cobimetinib or a pharmaceutically acceptable salt thereof per day.

31. The method of claim 30, wherein the subject is administered about 60 mg of cobimetinib or a pharmaceutically acceptable salt thereof per day.

32. The method of any one of claims 19 to 31, wherein cobimetinib or a pharmaceutically acceptable salt thereof is administered daily for 21 consecutive days of a 28-day treatment cycle.

33. The method of claim 32, wherein cobimetinib or a pharmaceutically acceptable salt thereof is administered on days 1 to 21 of a 28-day treatment cycle.

34. The method of claim 32, wherein cobimetinib or a pharmaceutically acceptable salt thereof is administered on days 3 to 23 of a 28-day treatment cycle.

35. The method of any one of claims 19 to 34, wherein the subject is administered from about 500 mg to about 2000 mg, from about 500 mg to about 1000 mg, from about 750 mg to about 1000 mg, from about 750 mg to about 2000 mg, from about 1000 mg to about 2000 mg, from about 1500 mg to about 1750 mg of the atezolizumab.

36. The method of claim 35, wherein the subject is administered about 840 mg of the atezolizumab.

37. The method of claim 35, wherein the subject is administered about 1680 mg of the atezolizumab.

38. The method of any one of claims 19 to 37, wherein the subject is administered atezolizumab every 14 days of a 28-day treatment cycle.

39. The method of any one of claims 19 to 37, wherein the subject is administered atezolizumab on days 1 and 15 of the 28-day treatment cycle.

40. The method of any one of claims 19 to 37, wherein the subject is administered atezolizumab every four weeks of the 28-day treatment cycle.

41. The method of claim 40, wherein the subject is administered atezolizumab on day one of the 28-day treatment cycle.

42. The method of any one of claims 19 to 37, wherein the subject is administered about 1680 mg of the atezolizumab on day one of the 28-day treatment cycle.

43. The method of any one of claims 19 to 42, wherein the subject was previously administered a course of treatment with an anti-PD-1 drug or anti-PD-L1 drug.

44. The method of any one of claims 19 to 43, wherein the subject experienced disease progression after treatment with immunotherapy, BRAF V600E therapy, or a combination of immunotherapy and BRAF V600E therapy.

45. The method of any one of claims 19 to 44, wherein said method for treating cancer is characterized by the absence of the development of squamous cell carcinoma in the human subject.
